(19) United States
(12) Patent Application Publication
(10) Pub. No.: US 2026/0294961 A1
(43) Pub. Date: Oct. 1, 2026

(54) PLANET ATMOSPHERE GASES ENWRAPPED INTO COMPOSITE NANOMATERIALS WITH MEDICAL TREATMENT APPLICATIONS

(71) Applicants: ARIZONA BOARD OF REGENTS ON BEHALF OF THE UNIVERSITY OF ARIZONA, Tucson, AZ (US); HONORHEALTH RESEARCH & INNOVATION INSTITUTE, Scottsdale, AZ (US)

(72) Inventors: Frederic ZENHAUSERN, Phoenix, AZ (US); Joellen L. RUSSELL, Tucson, AZ (US); Michael GORDON, Scottsdale, AZ (US); Marta SOWINSKI, Tucson, AZ (US); Justin MOSER, Scottsdale, AZ (US); Ashlee HARRIS, Phoenix, AZ (US); Zhu ZHAO, Phoenix, AZ (US); Matthew BARRETT, Phoenix, AZ (US)

(21) Appl. No.: 19/481,523

(22) PCT Filed: May 3, 2024
(86) PCT No.: PCT/US2024/027766

§ 371 Date: Nov. 4, 2025

Related U.S. Application Data
(60) Provisional application No. 63/464,421, filed on May 5, 2023.

(51) Int. Cl. A61K 33/00; A61K 9/50; A61K 33/04; G01N 33/00

(52) U.S. Cl. CPC A61K 33/00; A61K 9/5068; A61K 33/04; G01N 33/0075

(57) ABSTRACT

Provided are methods of treating a biological system with a gas marble, including related methods of making a gas marble from an identified gaseous chemical compound from a planetary source, including a planetary atmosphere. The collected gaseous chemical compound can be activated with a liquid having an assembly of composite nanomaterials to form stable gas marbles that are, in turn, used with a treatment method. Also provided are stable marbles gases useful in applications related to biological systems, including health-related applications by medical gas therapy.

### CROSS-REFERENCE TO RELATED APPLICATIONS

This application claims priority from U.S. Provisional Patent Application No. 63/464,421, filed May 5, 2023, which is incorporated by reference herein to the extent not inconsistent herewith.

### BACKGROUND

Provided herein are methods and related devices and systems useful for identifying planetary gas and containing the identified planetary gas in a stable configuration for subsequent use, including in medical treatment applications.

Medical ventilation, analgesia or sedation are common medical procedures involving the delivery of gaseous substances. Gas and liquid entrapping into gel or foam materials have been provided for various applications, including drug delivery. Those methods and materials, however, suffer from issues related to ensuring the active gas component is stable, including for long-term storage. This is reflected by medical gas therapy conventionally being limited to inhalation delivery to the respiratory system. Such gas delivery for therapeutic and treatment fundamentally suffers from inaccurate dosage and safety issues.

Another approach is medical gas therapy (MGT) using biological or noble gases with therapeutic benefits (e.g. neuroprotection) which have often been limited to flowing the gas in the breathing circuit, presenting major challenges such as inaccurate set doses and safety. An emerging field of pharmaceutical grade pro-drugs might be a more effective strategy to enable traditional route delivery of medical gases (e.g., carbon monoxide-releasing molecules or CORM). However, the pro-drugs route has several drawbacks such as systemic toxicity of the activated compound, the paucity of data on biodistribution of prodrugs, bystander effects and production costs. More recent developments have been reported with preclinical evaluation of gas-entrapping materials (GEMs) using foams and hydrogels for the delivery of carbon monoxide (CO) through the GI tract. That materials processing and gas entrapping is not readily suitable for the preparation of gas mixtures and shelf-life storage of these GEM is uncertain. The preparation of those GEMs requires pressurization and high temperature processing to perfuse gases into the material's pores.

It can also be challenging to obtain desired gases at desired purity and quantities without unduly impacting costs, stability, and desired dosage. Planetary atmospheres are evolving and offer unexploited sources for discovery of light isotopes (e.g., volatiles on Mars) or gas mixtures having some therapeutic benefits. For example, NASA's James Webb Space Telescope captured definitive evidence of carbon dioxide in the atmosphere of a planet beyond our solar system. Using the Atacama Large Millimeter/submillimeter Array (ALMA) in the Chilean Andes, astronomers detected hot gas bubble swirling around the Milky Way's supermassive black hole. Other contributions may also come from gases seeping out from a planet's interior (e.g., ocean sediments or planet mantle-driven noble gases). As climate change impacts the composition of the Earth atmosphere, with its vast capacity, the Earth atmosphere is also a frontier for the discovery of future gas therapeutics.

Using advanced algorithms and computational tools, we can monitor the mechanisms of climate change, including studying the role of ocean winds. The exchange of noble gases between the ocean and atmosphere depends on temperature, winds, and mediation by bubbles. Current climate trends observed and simulated, reflect that warming and increased winds that will favor enhanced super-saturation conditions, and therefore provide opportunity for enhanced collection and/or mining of compounds associated with the Earth's atmosphere.

Thus, it can be seen from the foregoing that improved methods for enwrapping gas and liquid droplets of various concentrations and from a multitude of gases and mixtures thereof are needed, including from a planetary source where the collection of a chemical compound has been optimized to an atmospheric region to maximize amount of chemical compound collected. Provided herein are such methods and resultant gas marble compositions, including from readily available sources, such as the planet atmosphere.

### SUMMARY OF THE INVENTION

Provided herein are methods of treating a biological system with a gas marble, along with related methods, devices and systems that facilitate the discovery and collection of planetary gases and volatiles that are useful in a number of applications, including for medical gas therapy. The planetary gas can be processed into a gas marble configuration that is useful as a storage and delivery system to a biological system (e.g. cell, tissue, organ or an individual), including for targeted (e.g. tropism) or systemic delivery, in a manner that is controlled so that an appropriate dose of the gas is precisely delivered.

Provided are gas (or liquid) enwrapping methods with nanoparticles to envelop materials with liquid or gases into marbles which can sustain external forces and internal pressures greater than atmospheric pressure without changing shapes or sizes (e.g. expansion). These enveloped gases are referred herein as marbles or gas marbles. Also provided are algorithms for discovering planetary gases, devices for high throughput fabrication of these marbles and extraction techniques for accessing planetary gases or isotopes. Further placement of these marbles into foam or gel materials can also be used for the preparation of pills or suppositories for oral, vaginal or rectal administration. The proposed platform can be used for investigating various headspace from organic and inorganic materials including profiling atmospheric gases to discover potential biological effects from new gaseous species and mixtures thereof (e.g., noble gases isotopes). There are clinical applications of relevance in oncology, neuroscience, cardiovascular, gastrointestinal and other diseases. Modes of therapeutic deliveries can also comprise topical, systemic, ingestion, complementary medicine delivery (e.g. acupuncture) or other medical interventions known in the art.

The instant methods are particularly useful for providing gas marbles that are small marbles which can persist and withstand positive or negative pressure, such as up to 10 atmospheres, for any of a variety of gases. These gas marbles are suitable for pharmaceutics and galenical preparation for facilitating administration into a convenient application platform, such as a pill or a suppository. In this manner, the methods and related composition of matter and devices, provide the means for administering gas as a treatment in a manner that improves a therapeutic index, is cost effective and safe, without sacrificing gas marble quality. Therapeutic index may refer to an improved outcome, reduced risk, reduced susceptibility, improved delivery of a therapeutic, or the like.

Provided are methods method of treating a biological system with a gas marble by providing a gas marble containing a gaseous chemical compound useful for treating the biological system, wherein the gaseous chemical compound is from a planetary source. The gas marble is applied to the biological system, thereby treating the biologic system with the gas marble. Depending on the biological system and the desired treatment, the applying may be oral, rectal, vaginal, topical, intra-venous, Intraperitoneal, and the like, with the gas marble(s) formulated accordingly.

Provided are various methods for making a gas marble, including a gas marble that is to be used in a method of treating a biological system. The method may comprise identifying a chemical compound from the planetary source, preferably a gaseous chemical compound from a planetary gas. The planetary source may be from the Earth's atmosphere. The gaseous chemical compound is collected from the planetary gas. A gas container is provided in fluid communication with a liquid vessel that contains a fluid mixture having a selected physico-chemical property, wherein the fluid mixture is configured to support an assembly of composite nanomaterials. The collected gases are loaded into the gas container and activated in the gas container with the assembly of composite nanomaterials, wherein the assembly of composite nanomaterials are partially wet. In this manner, bubbles are generated in the fluid mixture and stable gas marbles are formed that contain the chemical compound.

The chemical compound can be in the gaseous phase and, therefore, is also referred herein as a “gaseous chemical compound.”

The selected physico-chemical property may be viscosity and/or resistance to evaporation. The property is selected to generate subsequent marbles having a desired property, such as related to stability, shelf-life storage time, gas concentration, marble size, marble shape, and the like.

The method may further comprise the step of optimizing a physical gas parameter, such as a gas mass, concentration and/or a gas temperature, for computing a gas flow and a gas spatial variation within a planet climate condition to identify a planetary geographic area having a high release of the chemical compound. This aspect is relevant for efficient collection of the chemical compound, including a desired gas in the planetary atmosphere. As a plant undergoes climate change there can be temporal and/or spatial variation in the presence and/or amount of a desired gas in the atmosphere.

The gaseous chemical compound can be from a planetary source that is the planetary atmosphere, and may be from a family of noble gases and/or isotopes thereof.

The planetary gas can be from any of a variety of sources, such as from Earth, an astronomical object in the Earth's solar system, or is identified from an analysis of an astronomical object in the Earth solar system (including a planet), or an extra solar (exo)-planet.

The method may further comprise the step of preparing a pharmaceutical formulation from said gas marble. For example, the pharmaceutical formulation may be provided as an oral delivery capsule, a suppository delivery capsule, a topical patch, a foam, a cream, or an intravenously administered formulation. The desired formulation depends on the location to which the gas is to be delivered. For a skin-delivery therapeutic, the formulation may be a patch, cream or lotion that is applied to the skin, with the gas marbles embedded therein.

The method may further comprise the step of providing a selected dose of the gaseous chemical compound in the gas marble, including the selected dose that is greater than or equal to 1 pg and less than or equal to 10 g.

The gas marbles may be contained in a biodegradable shell comprising a lipid, glyceryl dibehenate, glyceryl distearate, oligosaccharides components, a biopolymer, a functionalized porous silicon nanoparticle, a plant-derived nanoparticle, or a composite material or composite materials, so long as the biodegradable shell is capable of storing the gaseous chemical compound, for a time period and that can degrade to facilitate release of the stored chemical compound to the desired patient, including a specific biological location within or on patient. The biodegradable shell may comprise a functionalized nanolipid particle with a ligand, wherein the ligand is a peptide, a protein or a nucleic acid.

The gas marble may further comprise a solid excipient for controlled release of the chemical compound, including by rapid onset and/or prolonged release. Rapid onset may refer to release of chemical compound that occurs on the order of 1 hour or less, such as between a few seconds and a few minutes, upon introduction to a desired site. Prolonged release, in contrast, may refer to release of chemical compound that occurs over a time scale of about 1 hour or more, such as between about 1 hour and about one to seven days. The release by rapid onset may be induced by pH activation, electromagnetic irradiation (such as photon beam or ultrasonic irradiation) and/or enzymatic digestion.

The gas marble may further comprise an adjuvant. Examples include adjuvants useful for wetting, emulsifying, suspending, flavoring (e.g., sweetening), and/or perfuming. Techniques for making oral dosage forms are generally described in, for example, Modern Pharmaceutics, Chapters 9 and 10 (Banker & Rhodes, Editors (1979)). See also, Lieberman et al., Pharmaceutical Dosage Forms: Tablets (1981). See also, Ansel, Introduction to Pharmaceutical Dosage Forms (2nd Edition (1976)).

The gas marble may have a shell configured to withstand a physical force associated with an external environment and/or handling, including the physical force by one or more of gravity-induced drainage, liquid evaporation, presence of nuclei, Laplace overpressure.

The chemical compound may be associated with a planetary atmosphere undergoing a measurable and quantifiable perturbation, including by climate change, wherein the perturbation is measured and quantified by an aerial, oceanic or satellite measurement. This reflects that the chemical compound, beyond simply isolating from air or taken from a commercially-available gas composition, can be selectively targeted to take advantage of changes in the planetary atmosphere as the environment changes. Accordingly, the method may further comprise utilizing algorithms to identify a chemical compound in the atmosphere. For example, the method may further comprise the steps of: assessing, calculating and mapping outcomes based on said aerial, oceanic or satellite measurement with a mathematical computing algorithm to identify, locate and quantify said chemical compounds or a precursor thereof in said planetary atmosphere; and optionally storing said assessed and mapped outcomes onto a computer-readable database for subsequent review and use.

The method may further comprise the steps of determining an optimal gas release location on a planetary geographic area and timepoint range for collection of the chemical compound or a precursor thereof from the planetary atmosphere. This can provide economic benefit of maximizing collection by identifying where and when to collect the chemical compound. This determination may rely on remote sensing, such as by satellites, aerial and/or oceanic monitoring.

The method may further comprise the steps of: processing a first chemical compound or mixture thereof from the planetary atmosphere; applying the first chemical compound or mixture thereof to solubilize a second chemical compound from a planetary crust region and/or an oceanic fluid; releasing the solubilized second compound into the planetary atmosphere; and collecting said second chemical compound from the planetary atmosphere using a direct gas collection such as airborne, ship, buoy or other suitable sampling method, for example using a filter made of polymer fibers, nanoporous materials or a metal-organic framework.

The chemical compound may comprise NO gas, CO gas, noble gases or isotopes thereof, oxygen, ozone, and/or sulfur-containing compounds or a gaseous species used in a medical gas therapy.

Any of the gas marbles described herein may be used in a method of treating a biological system by providing a gas marble and applying the gas marble to the biological system. Accordingly, any of the gas marbles described herein may be provided as a formulation suitable for application to a biological system, including to change at least one state of the biological system. Accordingly, provided herein are methods of treating a biological system with a gas marble, including by applying any of the gas marbles described herein to the biological system. In this manner, the biologic system is treated with the gas marble. For example, the application may relate to a radiological incident, including a potential exposure to ionizing radiation from a multitude of modalities such as electrons, photons, neutrons or combination thereof.

The biological system may correspond to a cellular, sub-cellular, or molecular components, a tissue, or the body of a patient in need of a medical gas therapy, and/or a patient undergoing a multi-modal therapy comprising chemo, radio, cellular, gene, thermo or dynamic therapies, and/or an individual undergoing a physiological performance enhancement.

The patient may suffer from a cancer, an infection, an autoimmune disease, a neurological disease, a cardiovascular disease, a rare disease, a trauma, or a disease-induced environmental exposure, including irradiation.

The applying step may be by oral administration, suppository administration, subcutaneous injection, intramuscular administration, intravenous administration, topical application, or another delivery modality.

Provided are gas marbles made by any of the disclosed methods. Provided herein are stable gas marbles made by any one of the methods described herein, wherein the stable gas marble is configured to maintain size and shape for long term storage. For example, provided are gas marbles having a stability range on the order of between one hour to one year, including up to between 1 year to 5 years, with a physiologically active range (where the gas within the stable gas marble interacts with a patient and selected targets thereof) that is between 1 second to 24 hours.

Provided are devices for implementing one or more methods described herein to make a gas marble. Provided herein are systems, devices, and components thereof for identifying and collecting a chemical compound used in any of the methods described herein.

Without wishing to be bound by any particular theory, there may be discussion herein of beliefs or understandings of underlying principles relating to the devices and methods disclosed herein. It is recognized that regardless of the ultimate correctness of any mechanistic explanation or hypothesis, an embodiment of the invention can nonetheless be operative and useful.

### BRIEF DESCRIPTION OF THE DRAWINGS

FIG. 1A left panel is a N₂ gas marble with PS NP produced over 3 months in the laboratory. The right panel is an air gas marble (3 days) with cinnamon nanoparticles (cinnamonNP). FIG. 1B is a schematic plot of gas marble stability by plotting gas marble mass as a function of time.

FIG. 2 is a schematic flow-chart summary of gas marble preparation, including from planetary gases mining, with medical treatment application.

FIG. 3 is a summary of the ability to use the instant gas marbles as a drug to cause a change in state of a biological system.

FIG. 4 is a summary of steps for making gas marbles from a planetary gas and use of the gas in the gas marbles as a therapeutic or treatment on a biological system.

FIG. 5 is a schematic of a “5 syringe stand.”

FIG. 6 is a schematic of a “gas bubble generator.”

FIG. 7 is a schematic of a “gas bubble tank.”

FIG. 8 is a schematic of another embodiment of a “gas bubble tank.”

FIG. 9 is a setup with mass flow controllers and cell culture chamber to assess biological effects of gases on biological systems, including a cell culture.

FIG. 10 is a photograph of gas marbles packed within a capsule for controlled release of gas to a patient.

FIG. 11A is a photograph of Preciterol ATO-5, Solid lipid, Glyceryl Distearate, Pharmaceutical Ingredient that is commercially available at T=0. FIG. 11B is a photograph of lipid-based marbles at T=1 hr and FIG. 11C at T=3 hrs.

FIG. 12A is a visible light-responsive GRM via Fr-MnCO composite for release of CO to HEK293 cells. FIG. 12B is a NIR-responsive GRM that is MnCO-GON for release of CO with near-infrared (NIR) stimulation. FIG. 12C is ultrasound (US) stimulated release of NO by BNN6-SPION@hMSN. FIG. 12D is X-ray induced release of gas from PEG-USMs-SNO. FIG. 12E is photothermal-responsive GRM of NO via SPION@PDA@MSN-SNO. FIG. 12F is magnetothermal-responsive GRM.

FIG. 13A is a plot of contact angle of unmodified (pink) and modified (blue) chitosan particles. FIG. 13B is a plot of FTIR of unmodified (black) and modified (blue) chitosan particles. 13C is an electron micrograph image of a chitosan-derived gas marble. FIG. 13D is a plot of marble mass as a function of time for seven marbles, illustrating gas marble stability out to at least 10 days. FIG. 13E Change in H₂ concentration in response to exposure to H₂-filled marble. FIG. 13F H2-filled marble decreased ROS levels in skin cells exposed to 8 Gy X-ray followed by 1.2 ppm H₂. FIG. 13G: H₂-marble skin patch for dermal application.

FIG. 14A is a plot of gas marble mass as a function of time illustrating stability of a chitosan-derived gas marble out to 45 days or more. FIG. 14B is a plot illustrating effect of application of a CO gas marble entrapped using chitosan.

FIG. 15 is a gas marble formulation schematic with a representative image of a marble produced by this method with CO gas using chitosan or Cinnamomum cassia bark particles.

FIG. 16A-16D: Hydrogen gas ameliorates radiation damage in vitro. FIG. 16A Cell viability; FIG. 16B ROS intensity; FIG. 16C Quantification of DNA double-stranded break repair at 1 h post-IR and 24 h; FIG. 16D post-IR.

### DETAILED DESCRIPTION OF THE INVENTION

In the following description, numerous specific details of the devices, device components and methods of the present invention are set forth in order to provide a thorough explanation of the precise nature of the invention. It will be apparent, however, to those of skill in the art that the invention can be practiced without these specific details.

In general, the terms and phrases used herein have their art-recognized meaning, which can be found by reference to standard texts, journal references and contexts known to those skilled in the art. The following definitions are provided to clarify their specific use in the context of the invention.

As used herein, “family of noble gases” refers to inert gases of Group VIIIA of the periodic table, including helium, neon, argon, krypton, xenon, radon and oganesson, as well as their stable isotopes and radiogenic species. Of course, the methods and systems provided herein are compatible with any of a range of gases, including newly discovered gases. Of particular interest, are those gases having use in an application, such as a biological affect, therapeutic activity, treatment impact, a chemical process and/or a manufacturing process. Of particular interest are those gases that are associated with climate change, including gases having an increased release into the Earth's atmosphere.

As used herein, “planetary gas” is used broadly to refer to any chemical compound that is part of, or suspended in, an astronomical body's atmosphere. The gas may be in the atmosphere and/or released by a liquid and/or crust of the body. The planetary gas may be from the Earth. Similarly, “planetary source” is used broadly herein to refer to any portion of a planet that contains or generates a planetary gas, and particularly a gaseous chemical compound of interest, or provides a precursor of the gaseous chemical compound. Accordingly, the planetary source may be the planet's atmosphere, a liquid on or in the planet, the planetary crust, or any combination thereof.

“Physico-chemical property” refers to a property of a fluid mixture within a liquid vessel that is operably connected (e.g., in fluid communication) with a gas container. Examples include, but are not limited to, viscosity, resistance to evaporation, boiling point, vapor pressure, density, and the like.

“Composite nanomaterials” refers to a material useful for forming stable gas marble, such that the chemical compound, including a gas compound, can be stored long-term and at a reliable dose for subsequent biological applications. Examples of components of composite nanomaterials include, but are not limited to, polymer nanoparticles such as polystyrene beads, Polyethylene glycol or polyethylene oxide (PEG, PEO) and derivatives, lipids such as solid lipids formulation, liposomes or nanolipid particles (NLPs), dendrimers, but also nanoparticles made of silicon, gold, silver, iron, bimetallic gold and silver, magnetic particles or other materials, including nanocrystals such as quantum dots, and carbon nanotubes, or many other types of nanomaterials that can be used for conventional drug delivery depending on the specific application and requirements. Composite nanomaterials are wettable to facilitate bubbling of collected gas having the desired chemical compound from which stable gas marbles are generated.

“Stable” is used herein to describe gas marbles. Stable refers to the gas chemical compounds in the gas marble that maintains efficacy for a biological treatment for a desired storage time and treatment time. Examples include up to about 1 year to 5 years. For a chemical compound that is a gas, at least 80% of the mass of the gas remains stable over the storage time, including a storage time ranging from greater than 5 days and less than 3 months, and any subranges thereof. Stable reflects that the gas marbles have a stable shape and/or size, and is optionally more quantitatively characterized in terms of maintaining a shape and/or size over a storage time, such as between 5 days and 3 months, including a deviation from original size (e.g., volume) or shape (e.g., average diameter) that is less than 10%, less than 5% or less than 1%.

“Physical gas parameter” refers to a parameter that can be optimized or identified to inform a planetary geographic area selection corresponding to a high release rate. For example, a gas mass or concentration and/or a gas temperature can be measured with localized maximums identified. This can then inform where the chemical compound should be collected, thereby maximizing collection efficiency.

With respect to a shell of the gas marble, a “composite material” refers to a material formed from at least two compounds. Examples include, but are not limited to, a combination of lipids nanomaterials (e.g. phospholipids with cholesterol or solid lipids like glyceryl distearate functionalized with a ligand such as a peptide, nucleic acid, immunoglobulin to target a biological system (e.g. a cell, lung or gut epithelium) for interactions with said gas in view of triggering some biological effects.

“Algorithm” refers to a series of computationally-implemented steps used to inform collection of a planetary gas, and more particularly, the desired chemical compound in the planetary gas that will be incorporated within the gas marble. These algorithms can comprise statistical models (e.g. regression, clustering, classification, time series, ensemble models, neural networks and Bayesian models), machine learning algorithms (ML), dynamical systems theory, data assimilation techniques, regional climate models (RCMs), Earth system models (ESMs), general circulation models (GCMs). Other techniques for analyzing cosmological atmosphere of planets or exo-planets can comprise using retrieval algorithms, transit spectroscopy algorithms, eclipse spectroscopy algorithms or other methods known by those skilled in the prior art.

The methods and devices/systems provided herein are compatible with range of chemical compounds, including any gaseous species used in medical gas therapies.

“Planetary geographic area” refers to a location having higher than average gas release and, therefore, larger relative amount of the desired chemical compound. For example, as any one or more of ocean currents, ocean temperature, ocean pH change, including in a spatially-varying manner, there can be increased localized gas release from the ocean. This can change depending on the season, so that there is a “timepoint range” associated with an identified planetary geographic area having a localized maximum or maximum corresponding to “optimal gas release.”

As described and discussed, the gas marble is useful for treating a biological system. “Biological system” is used broadly to refer to a living material (e.g. cell, tissue, organelle, organ), including in vivo, in vitro, or ex vitro (e.g. organoids), that can respond to a gas therapy. This can include to treat an adverse disease state or condition. This can include a preventative treatment. This can include a treatment to improve or enhance biological function, including in a normal individual, such as for a “physiological performance enhancement” wherein the biological system is effectively improved based on one or more physiological parameters.

“Patient” is used broadly herein to refer to an individual that is treated with the marble gas. Patient may be a non-human animal or a human.

### Example 1: Gases-As-Drugs Delivery Platform Technology

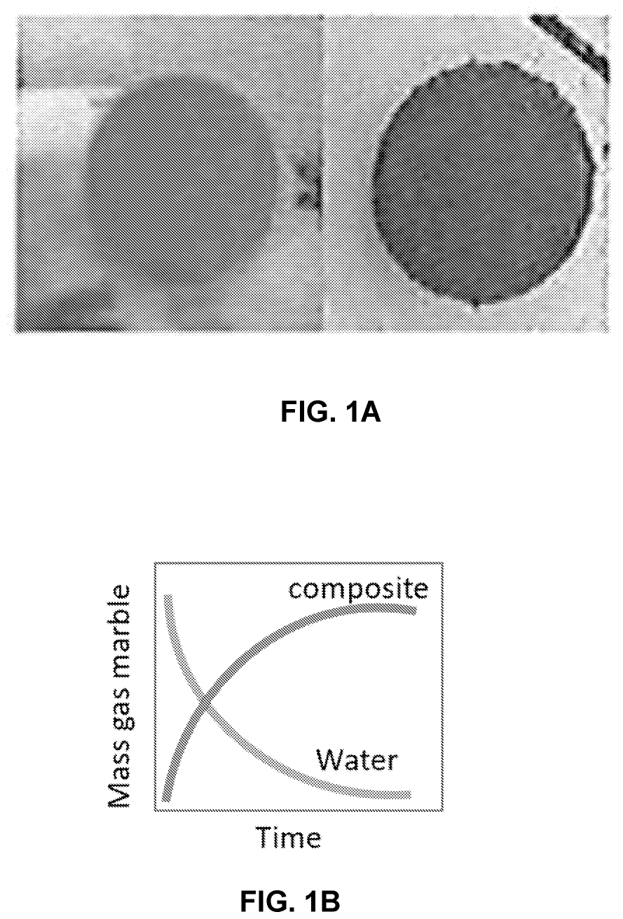

This example demonstrates (1) gas marbles useful for trapping nitride oxide and/or carbon monoxide bubbles into a stable formulation for future delivery of NO/CO through the GI tract (i.e. suppository) of a small animal model, and (2) apply cutting edge computational models to assess planetary mechanisms of ocean crust production; exchange of gas isotopes with the Earth atmosphere; and extrapolate to gas planet mining to determine potential gaseous species as therapeutic candidates. FIG. 1 illustrates a gas marble formed with N₂ gas with a PS shell (left panel) and an air gas marble with cinnamon nanoparticles (cinnamonNP) (right panel).

First, we formulate various nanomaterials including lipid-based particles (e.g. plant-based vesicles) and other biodegradable shells (e.g. glyceryl dibehenate or glyceryl distearate) for the gas marbles (e.g. NO and CO gases), and then incorporate them into solid excipients (e.g., Suppocire hard fats for suppository) for rapid onset (e.g., pH activation; enzymatic digestion) and/or prolonged (controlled) release (e.g., ultrasounds). Melting temperatures and combination of adjuvants for wettability are tested. For mice studies, gas marbles dosage of 0.5-5 g/kg can be formulated. Materials characterization is performed by fluorescence and electron microscopy, while gas quantification in the marbles is performed using a gas chromatography-thermal conductivity detector system (GC-TCD).

Since they are chemically inert and respond to physical factors and bubble-enhanced diffusivity differently, noble gases are introduced into ocean-only and coupled climate and earth system models. Numerical simulations of modern and future climate with medically useful noble gases are performed and assessed to determine optimal locations and seasons for enriched ocean conditions to extract. In addition, saturation anomalies of noble gases have been linked to bubble-related diffusivities which are projected to change as ocean winds and waves continue to increase.

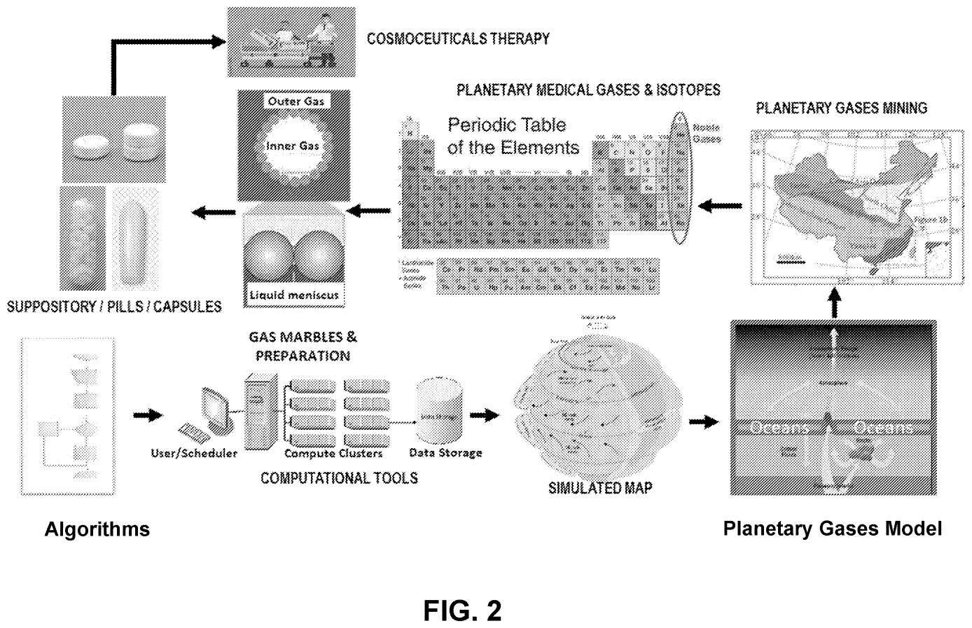

As summarized in FIG. 2, this example validates: (1) a formulation of gas marbles for long lasting storage of CO/NO that can be formulated into a suppository for future administration in a small animal; and (2) a modeling algorithm to identify potential planetary routes, locations, and seasons for candidate gaseous species.

### Example 2: Medical Gas Therapy & Atmospheric Gases Drug Discovery

This example is directed to the development of gas/liquid marbles to study gas therapy and therapeutic delivery using novel mixtures from the planet(s) atmosphere. Herbal therapeutic aerosol delivery dates back to the ancient Egyptians. Currently, only oxygen therapy is applied to patients with some ozone therapy by intramuscular delivery in pain patients. There remains, however, dosage issues along with controversy with respect to therapeutic effectiveness.

There are two general categories: (i) biological gases (e.g. CO, NO, H₂S, O₂ . . . ) and (ii) noble gases (e.g. Xe, Ar, He . . . ). MGT has demonstrated multiple tissue protective effects in vitro and in small animal models. The delivery platforms may be categorized as: (i) inhalation or (ii) pro-drugs (and combination with Nanoparticles). Radioprotective effect of xenon inhalations and other organo-protection of Xe and Ar are demonstrated. CO involved in Nrf2/HO-1 (heme oxygenase) signaling pathway and pain management.

Biological mechanisms of medical gases have been examiner. See, e.g., Zafonte et al, 2022. Although putative biological effects have been identified, there is a fundamental lack of multi-omics characterization including with NxGen and RNA SEQ, Gen editing, etc. Furthermore, there are no reported combinatorial MGT effects.

There are challenges related to MGT, including a need for new models that study the effects of multi-MGT via multiple gas administration. It is also challenging to control gas concentration for direct inhalation studies and avoid offsite deleterious effects. Pro-drugs can be a more effective strategy to enable traditional route delivery of medical gases (e.g., “CO in a pill”, CORM and Xe-ELIP). Nitride Oxide (NO) can be utilized for in situ tumor ablation. With this in mind, the Earth atmosphere, having a vast capacity, can be the newest frontier for the discovery of therapeutics, including neurotherapeutics. This aligns with the broad medical applications of gas therapy.

Light isotopes volatiles have been detected on Mars (e.g. krypton). NO has roles in the regulation of blood flow and thrombosis, neurotransmission, the normal functioning of the genitourinary system, and the inflammation response and host defense. Nitrates and nitrites can be bioactivated into NO and have also beneficial effects like lowering blood pressure but mechanisms still unclear.

Gas Marbles Carriers based Pharmaceutical Formulations: Aims include: 1) Formulating various nanomaterials, including “lipid-based” gas marbles and incorporating them into pills or suppository_base for rapid onset and/or prolonged (controlled) release. There are reports of gas entrapping materials (GEM) with protective effects. Accordingly, another aim is 2) Assessing “Gas-As-Drugs” and mixtures thereof by exploring gaseous species/isotopes from a planet atmosphere (e.g. Earth) which has/is/will evolve (e.g. climate change), opening a new era for “Planetary-based Gas Therapy Discovery” toward “CosmoCeuticals.”

Technological components are available for the gas marble generation. For example, capsule size is selected depending on species and weight, which impacts the desired gas application amount. On the gas discovery side, air sampling and collectors can be deployed via a drone. Chang et al. (2016). An AirCore sampling process can be used to sample and characterize atmospheric gases, including by a long (>100 m) and narrow (<1 cm) stainless steel tube that can retain a profile of atmospheric air. O. Membrive et al. “AirCore-HR: a high-resolution column sampling to enhance the vertical description of CH₄ and CO₂.” Atmos. Meas. Tech., 10, 2163-2181 (2017).

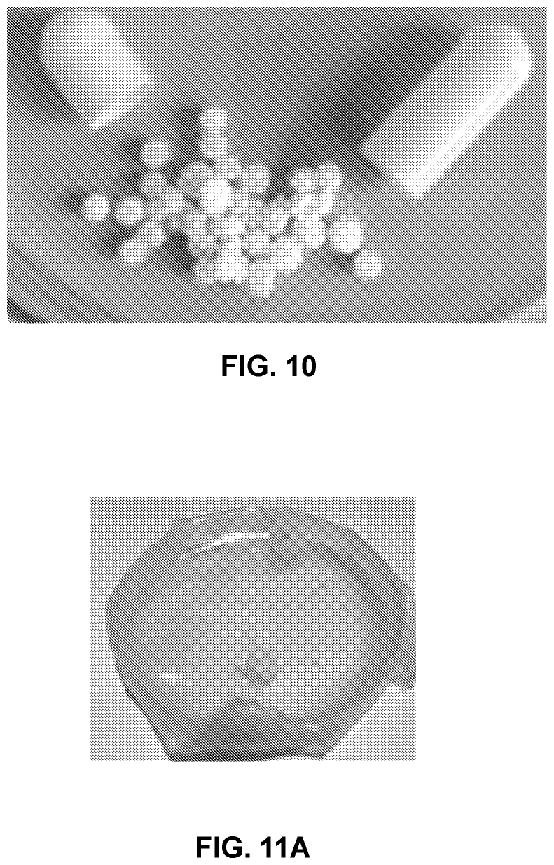

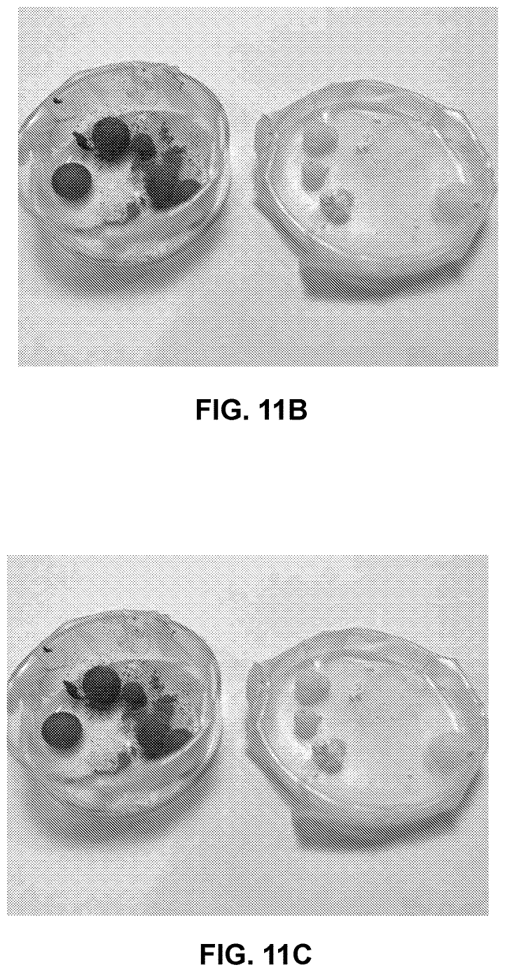

FIG. 10 illustrates a therapeutic package, with encapsulated gas containing materials. FIGS. 11A-11C are photographs of lipid-based marbles at different time points (0, 1 h and 3 h, respectively using cinnamon and solid lipid particles). The shell comprises Preciterol ATO-5, Solid lipid and Glyceryl Distearate. Cell cultures can be exposed to a variety of gas marbles.

### Example 3: Planetary Gas-As-Drugs

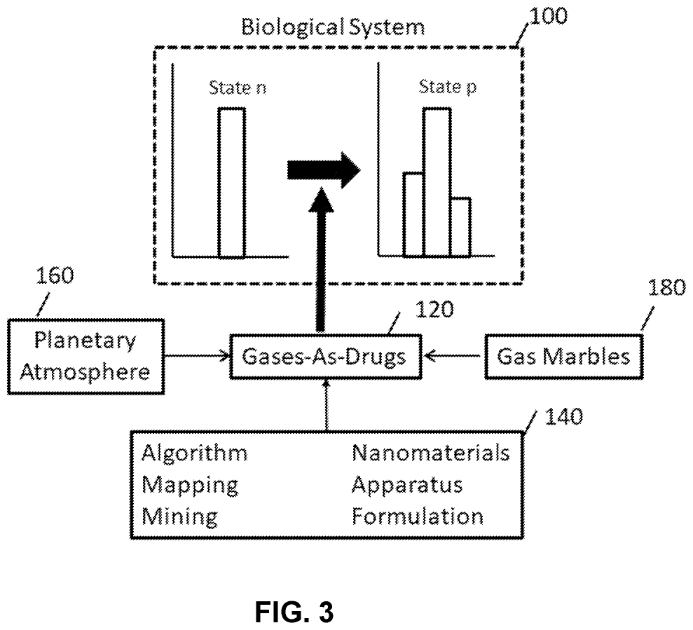

FIG. 3 illustrates the principle of the instant methods, wherein a biological system (100) in a state n (e.g. unstable) that can convert into a state p (e.g. stable) when triggered by gases-as-drugs delivery (120). The gases-as-drugs are prepared by using a toolbox (140) combining tools for discovering planetary atmospheric compounds (160) using algorithms and mining techniques as sources of gases for their trapping into stable gas marbles (180) during the materials preparation using a novel apparatus and methods for targeted deliveries that can impact various biological signaling pathways and tissues that can lead to the conversion of states in the biological system, for example treating an illness.

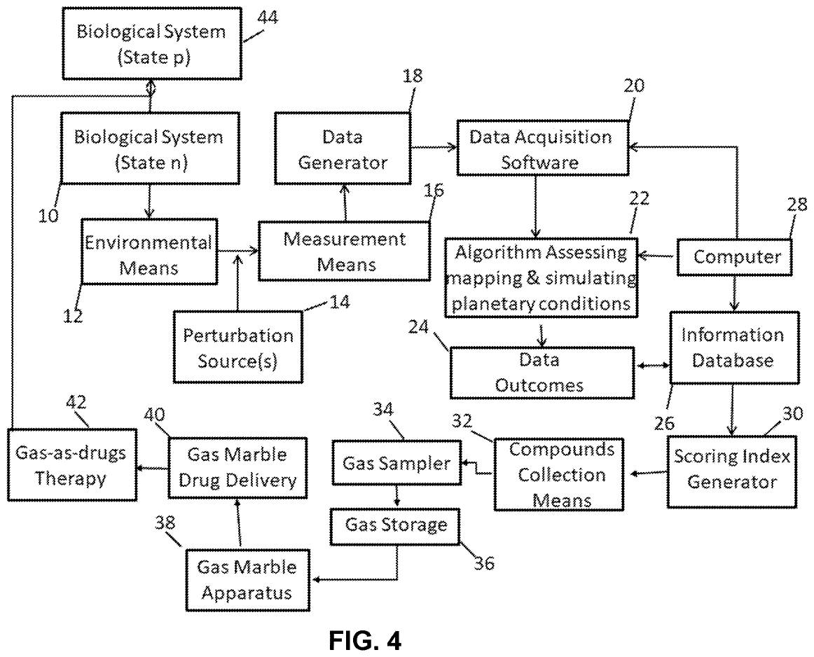

FIG. 4 is a schematic overview illustrating the process from a biological system in a state n (for example “ill” or unstable state) which can be converted into a state p (e.g., “health” or stable state) by application of the gas marbles described herein, including as made by any of the methods described herein. The workflow of the conversion may comprise the steps summarized in FIG. 4. First, a modification of an environmental condition under a planetary perturbation can be measured by physical probes and an algorithm generating data mapping of a probable outcome of said environmental conditions enhancing identification and/or access of chemical compounds, preferably in gaseous phases, which can be collected preferably from a planetary atmosphere. Second, an apparatus comprised of at least one gas container interfaced with a liquid vessel that can hold a fluid mixture of a determined physico-chemical property (e.g. viscosity) whose surface can support a raft of an assembly of nanomaterials. The previously collected gas(es) can be loaded into the gas container which after controlled activation can generate bubbles into said liquid reaching the surface under said raft at which interface a stable gas bubble can be rolled into the nanomaterials, including composite nanomaterials, forming gas marbles containing said gaseous compounds from said planetary atmosphere. Third, the gas marbles can be prepared into a pharmaceutical formulation such as an oral delivery capsule, a suppository delivery to be inserted into the rectum, vagina, or urethra of an animal or an individual. Other formulation techniques known by those skilled in the art, including nano-lipid shell materials, viral particles, mammalian or plant exosomes, and other targeted deliveries to specific tissues (e.g., lung or gut epithelium) or through systemic drug delivery infusion, can also be suitably encapsulated and delivered by delivery means in combination with the gas marble preparation and delivery of gases-as-drugs. Fourth, dosing of gases and mixtures thereof, and materials characterization can be performed using conventional analytical techniques such as by microscopy (e.g., electron microscopy, SEM, TEM), spectroscopies (e.g. FT-IR, UV spectrophotometry) or chromatography (e.g. GC, MS). Biological effects can be tested using multiple molecular and cellular techniques, including omics analyses (e.g., transcriptomics, proteomics, metabolomics, interactomics), biochemical, cell-based, microbial or imaging assays

In the embodiment of FIG. 4, a biological system in a state n (10) is exposed to an environmental means (12), for example a planetary atmosphere which can be undergoing perturbation (14) such as a climate change (e.g. CO₂ emissions, planetary winds) that can be measured by aerial, oceanic, or satellite measurements (16), for example. The measurements can be processed by a data generator (18) in communication with a data acquisition software (20) feeding data information to a simulation algorithm (22) to assess and map data outcomes (24) identifying, locating and quantifying chemical compounds in said planetary atmosphere which can be recorded and stored into a database (26) through a computer interface (28). For example, the maps generate a scoring index (30) that can guide the collection of noble gases compounds (32) which can be released from the planet Earth crust by the effect of deeper CO₂ concentrations in the oceans mining these gases from the water to the surface of the oceans where increased concentrations and/or various types of gases (e.g. isotopes, mixtures thereof) can be extracted and collected in situ through a gas sampler (34) and storage/transportation device (36), or other means known from those skilled in the art of geoscience and atmospheric measurement techniques. Gases are used as sources for the gas marble apparatus (38) for the fabrication of gas marbles which can then be formulated into pharmaceutical drug delivery means (40) to develop precise therapies of gases-as-drugs (42) that can drive the conversion of a biological system in a first state (e.g. ill state) to a second state (e.g. healthy state) (44), for example. The conversion can comprise treatment of disease signaling pathways involved in cancer, infection, auto-immune diseases, neurological, cardiovascular, or other diseases for which such a targeted and controlled gases delivery may provide beneficial biological effects. Clinical use of gas marbles-as-drugs can be applied to a broad range of healthcare applications from prevention, prognosis to treatment. For example, an embodiment of the present invention could provide medical countermeasures and biological protection from space or other sources of ionizing radiations. In another embodiment, topical treatment of skin diseases (e.g. melanoma, acne, eczema) could be releasing nitric oxide (NO) or other treatment gases. Organo-protection including neuroprotective effect under stress conditions can be efficiently performed by noble gases treatment (e.g. Xe, Ar).

### Example 4: Release of Gas from Gas Marble Packages

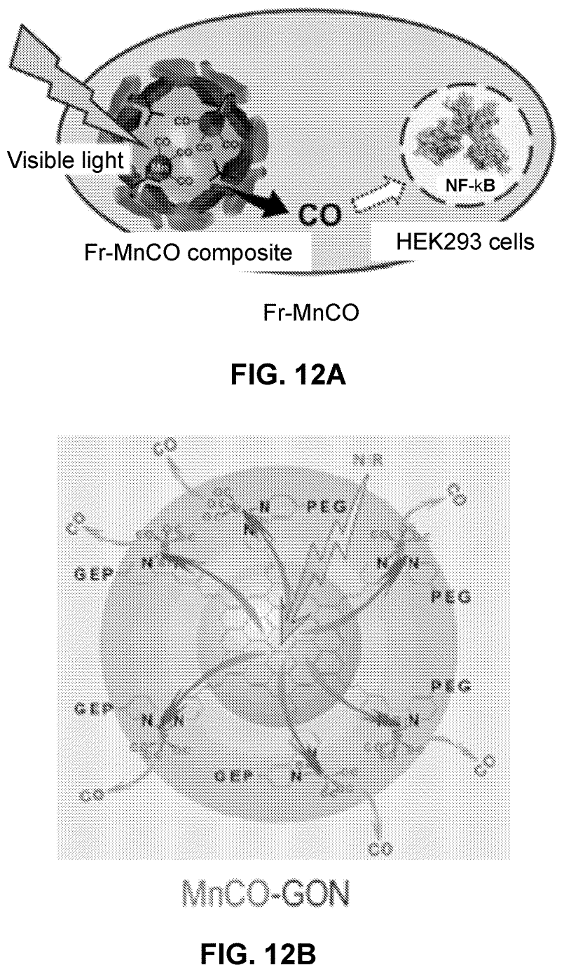

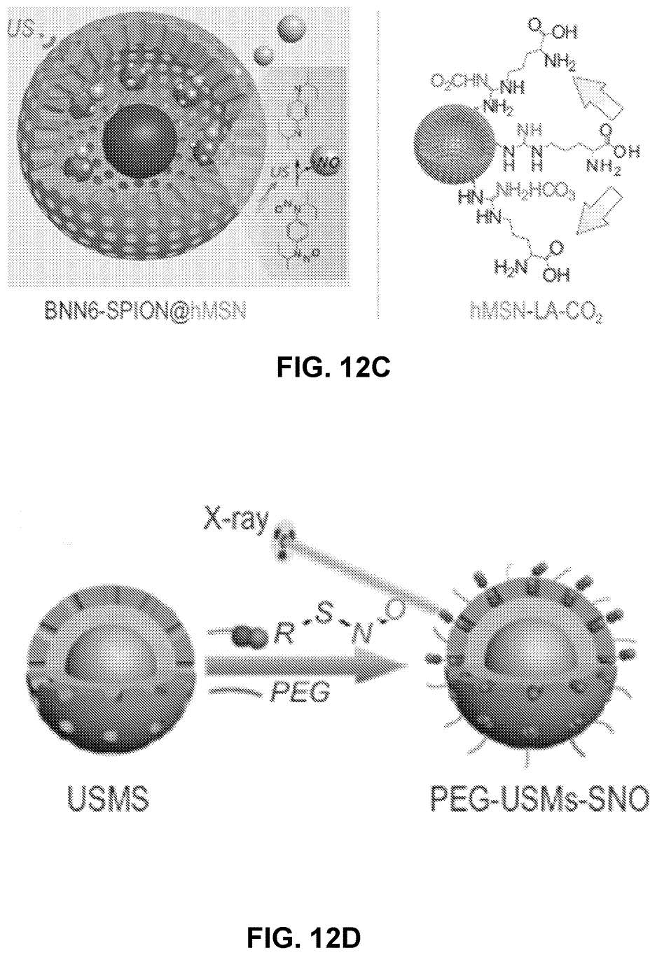

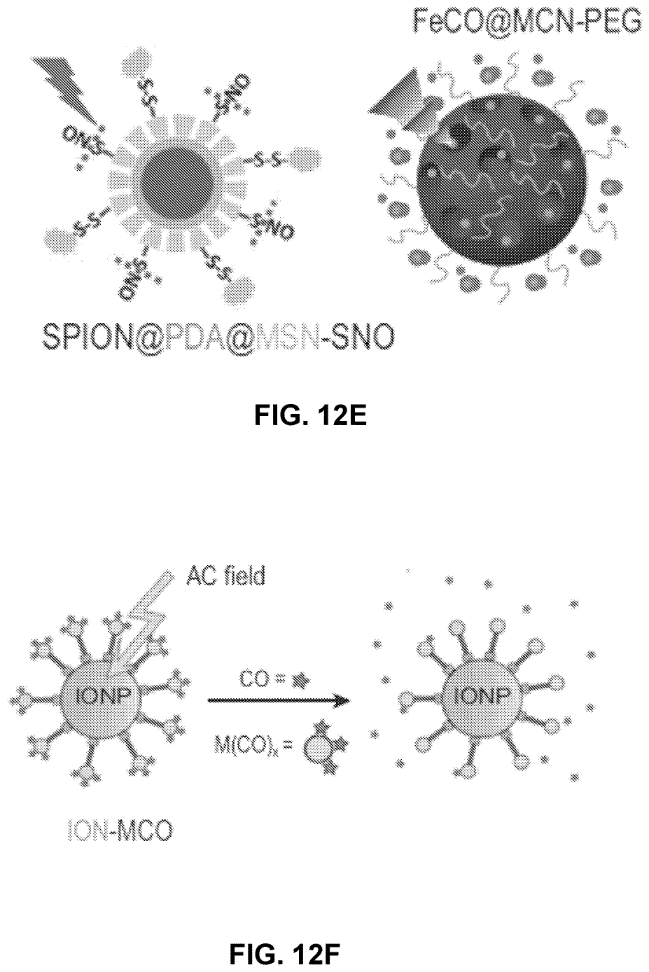

Referring to FIGS. 12A-12F summarizes various gas-releasing medicines, having different release mechanisms, such as: visible light-response gas releasing medicine (GRM) (FIG. 12A); NIR-released GRM (FIG. 12B); ultrasonic (US)-released GRM (FIG. 12C); x-ray released GRM (FIG. 12D); photo-thermal released GRM (FIG. 12E); magneto-thermal released GRM (FIG. 12F).

### Example 5: Systems and Devices

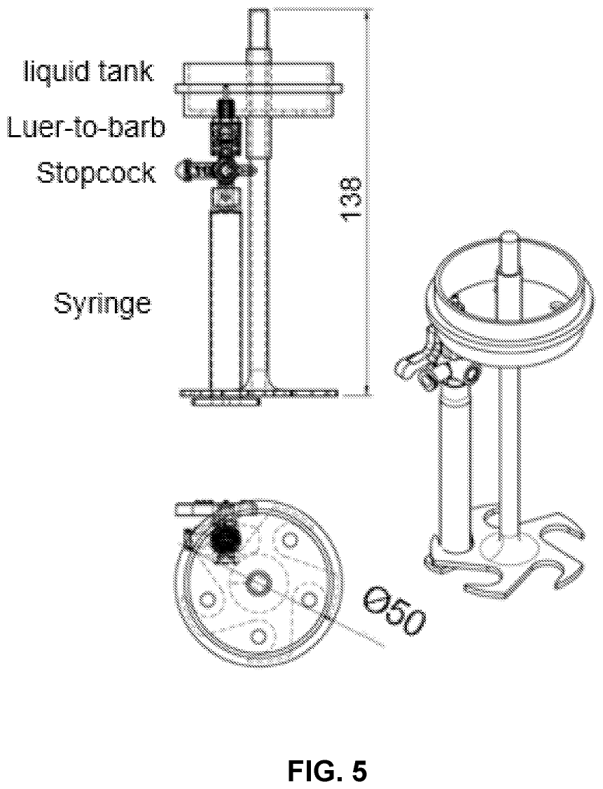

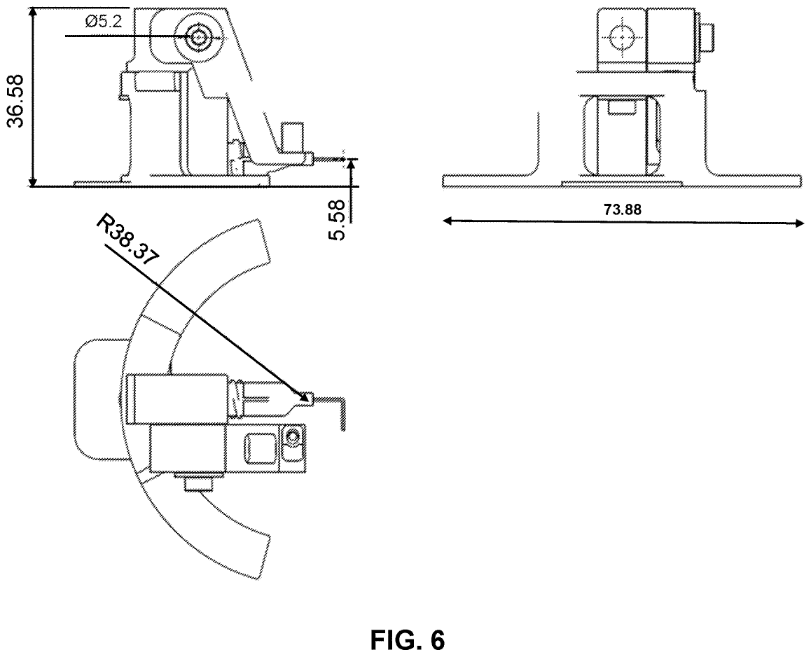

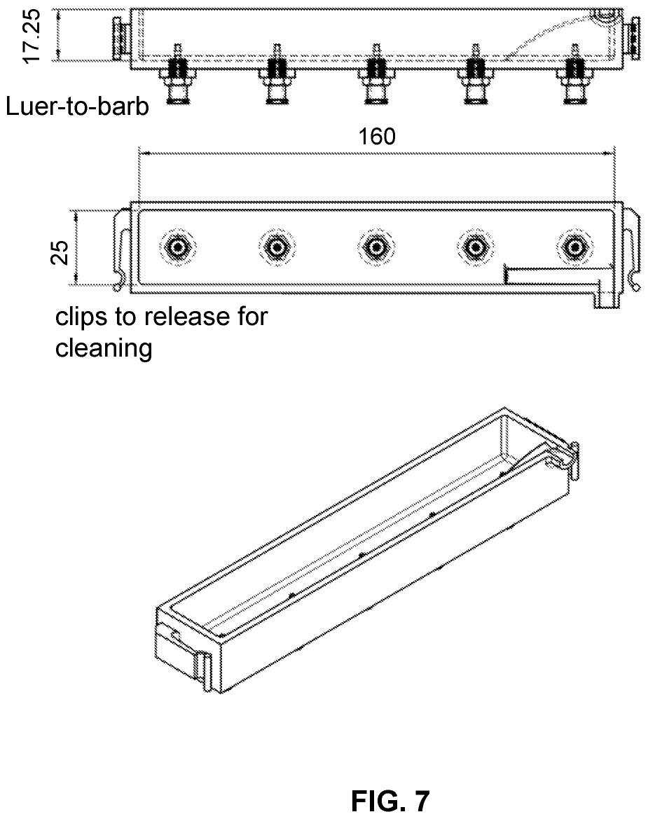

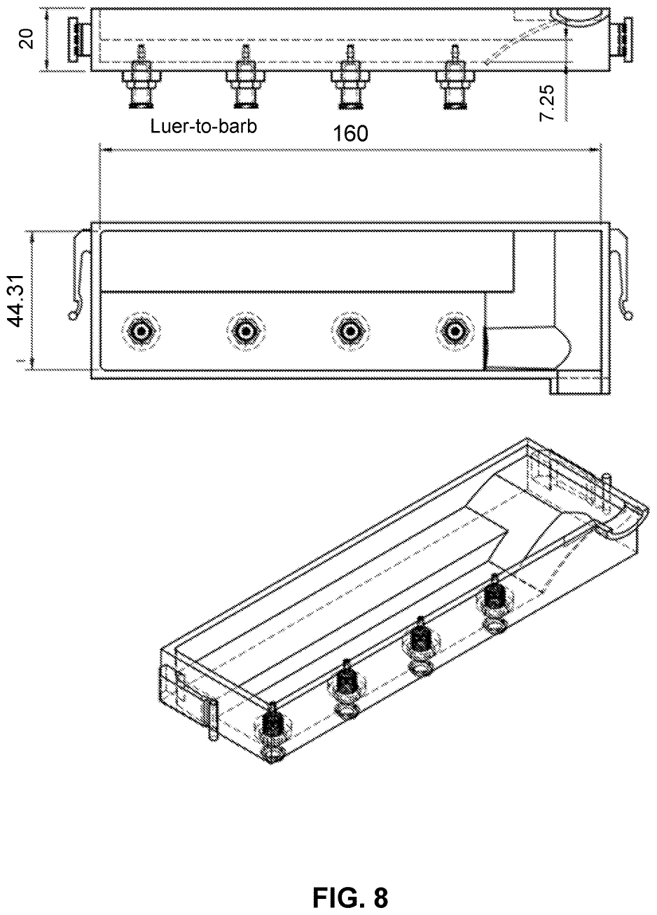

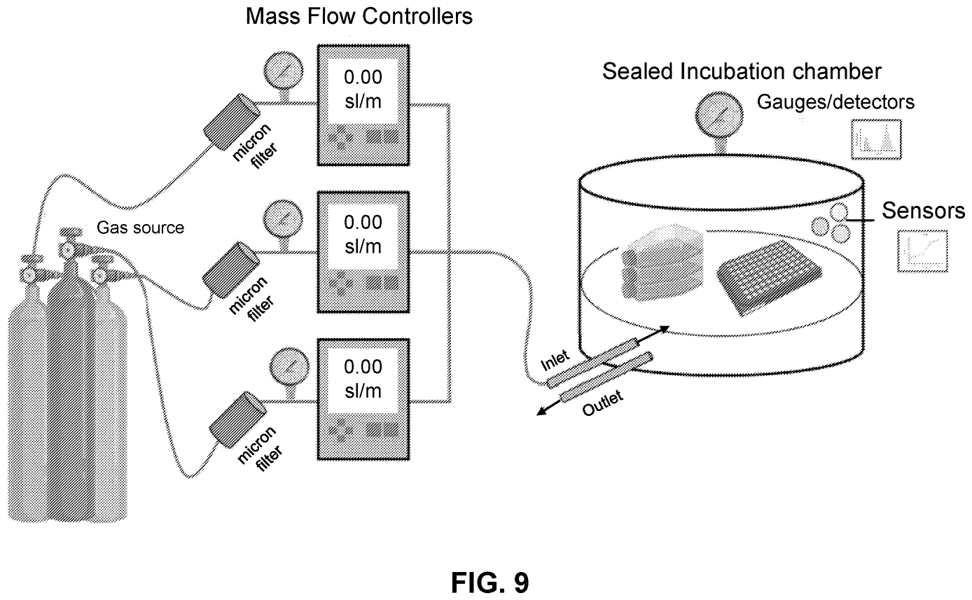

FIGS. 5-9 are schematic drawings of various components useful in characterization and development of the instant gas marbles, including an experimental setup to test the biological effects of gases on cells in culture. FIG. 9 illustrates an experimental set-up to evaluate effects of gas on cultured cells. Each gas flows from their cylinder source through micron filters, past inline pressure gauges and into the mass flow controllers (MFCs). MFCs accurately measure gas by mass (weight) which is unaffected by external conditions (temperature and pressure). From this measurement, gas flow is automatically controlled. The accurately measured gas flows out of the MFC and enters the incubation chamber to combine with other gasses. Once the ideal mixture is achieved, the chamber is sealed. Gas exposure and detection are initiated.

### Example 6: Gas Marble Therapeutics for the Mitigation of Radiation Injury

There has been a growing interest in utilizing gaseous molecules for therapeutic advancement as they exhibit promising abilities to reduce inflammation, regulate oxidative stress, provide cellular protection, and facilitate wound healing. However, delivery in this domain has predominantly centered around inhalation-based methods, which are hampered by challenges with dosing accuracy. As a result, many gasotransmitters, noble gases, or traditional gases have not yet been tested for their ability to mitigate radiation induced injuries. Recently, gas marble technology has emerged as a stable formulation by which to capture and contain gaseous species. The technology is made possible by the addition of partially wetted micro/nanoparticles to the air-liquid interface of liquid entrapped gas. As this fortification strengthens resistance to mechanical stress and provides remarkable stability it is called a gas marble. These robust preparations serve as an effective means of controlled delivery, encapsulating therapeutic gases to function as medical countermeasures against radiation exposure. This example is for the investigation of medical gases as MCMs to mitigate and/or treat injury to normal skin cells arising from exposure to ionizing radiation in human tissue in vitro models. In addition, the gas marble delivery system can be tested in vivo for its ability to deliver radiomitigating H₂ gas to ameliorate cutaneous radiation injury. With this in mind, therapeutic gas(es)/mixtures can be screened for their ability to elicit a radiomitigating effect following cutaneous radiation injury. In this manner, in vivo feasibility of the gas marble technology for the skin delivery of H₂ to mitigate radiation injury is examined.

Nuclear or radiological emergencies will necessitate an urgent mass casualty response where swift deployment and distribution of medical countermeasures (MCM) will be required to minimize tissue damage, hasten tissue recovery, restore physiological function, and improve patient survival. Bodily organs with rapid cell turnover and in direct contact with the external environmental, such as the skin, are most susceptible to significant damage as the exposed DNA might not have time to repair, leaving the cell vulnerable to mutations and apoptosis, resulting in barrier dysfunction (DiCarlo). To date, there are no FDA approved MCMs for cutaneous radiation injury and current curative effects remain unsatisfactory 1. Thus, there is a need for novel treatments to enhance preparedness and ensure a robust response to unexpected events.

Medical gas therapy in the form of gasotransmitters (e.g., CO, NO, H₂S), noble gases (e.g., He, Xe) or traditional gases (e.g., O₂, O₃) has the ability to treat injury or disease by modulating bioenergetic homeostasis, reducing oxidative stress, providing cellular protection, or promoting wound healing while improving patient recovery times2. Specifically, we and others showed that, in response to radiation exposure, molecular hydrogen (H₂) has been demonstrated to inhibit free radicals, modulate inflammation, and prevent apoptosis³. Other gases that have shown the ability to reduce oxidative stress, either alone or in combination, have not yet been tested for their radiomitigating ability. However, gas delivery in vivo has been limited primarily to inhalation as delivery attempts by gas releasing molecules suffer from inaccurate dosing and variable activation. Thus, a new, stable gas delivery system is needed.

Recently, it has been shown that liquid film entrapped gas can be fortified with micro/nanoparticles to generate “gas marbles” which are stable for over one year^{4,5}. With two liquid-gas interfaces providing superior mechanical strength, these gas-containing marbles can oppose escaping gas at pressures up to 10 times the Laplace pressure⁴. Clinically, these highly resistive marble formulations could be used as a controlled delivery vehicle in which to entrap therapeutic gas(es) to serve as MCMs to radiation exposure.

Using normal cells in vitro, medical gases and medical gas mixtures are tested for their ability to mitigate radiation induced cellular damage. Parameters such as cellular viability, DNA damage repair, and oxidative stress markers are evaluated. Optimal dosage regimen of each gas (ppm) are assessed, in addition to short and long timepoints, which include, 24 and 48 h acute post-exposure to X-rays.

Rodents with acute cutaneous radiation injury are exposed to radiomitigating H₂-filled marbles to assess their radiation mitigation efficiency. Tissue samples are collected for investigation of oxidative, inflammatory, and cell death pathways. Based on preliminary results, additional gas groups are added. Results inform subsequent large-scale in vivo study.

In the event of accidental exposure to ionizing radiation, the skin is the first line of defense from external insult, significantly compromising its function. The resulting damage plays a vital role in the progression of the injuries and patient survivability1,6,7. Within the skin, the melanocytes, hair follicles, stem cells, and basal keratinocytes are the most radiosensitive due to their high rate of cell division. Radiation exposure causes overwhelming oxidative stress and antioxidant imbalance resulting in erythema, edema, desquamation, ulceration, and ultimately barrier dysfunction1,8. The chronic inflammation impedes wound healing, leading to fibrosis. While several MCMs have been developed to address hematopoietic complications arising from radiation exposure, there are not yet approved MCMs to address skin injury. Worse, treatments address the symptoms without efforts toward restoration of function9.

Gaseous molecules have been increasingly explored for therapeutic development. In particular, molecular hydrogen (H₂) is a powerful antioxidant, known to reduce reactive oxygen species (ROS), including the hydroxyl radical (·OH)3,10-12. Generated extensively by radiolysis upon radiation exposure, ROS is a primary contributor to radiation-induced cell injury, exacerbating DNA damage and perpetuating oxidative stress13. As the hallmarks of cutaneous radiation injury include redox imbalance, inflammation, and apoptosis, H2 has been investigated for its ability to serve as an MCM mitigating radiation-induced skin damage14-16. Murine models treated with H2 experienced accelerated wound healing with reduced healing time and reduced malondialdehyde (MDA), superoxide dismutase (SOD), and IL-6 levels compared with the non-treated control14,15. In addition, other pharmaceutical gases (e.g., NO, CO, H2S, O3, O2, Xe) have demonstrated efficacy in treating wound healing17,18, oxidative stress 19,20, inflammation21,22, apoptosis23,24, and disrupted blood vessel integrity 25,26.

Favorably, many of these gases, including H2, are widely available for a low cost. Their use can circumvent the high expense and long development times associated with new pharmaceutical candidates27. However, translation has largely been limited due to the challenge of gas delivery28. Traditional delivery methods provide the gas for patients to inhale using a ventilator circuit, facemask, or nasal canula2. Cutaneous H2 delivery in vivo is limited to saturated H2-water made fresh for each application and delivered via spray bottle or, alternatively, inhalation chambers supplemented with ~1% H2. Those approaches result in inaccurate dosing which has been shown to vary based on inhalation method employed and even from patient to patient2.

To overcome this challenge, recent studies have explored the use of gas marble technology4,5,29 where gas is entrapped by a liquid film (e.g., a bubble). Normally, at atmospheric pressure, gravity induced drainage would cause this bubble to spontaneously burst and release the entrapped gas. However, by adding partially wetted particles to the air-liquid interface, the bubble becomes fortified and able to maintain its integrity (without change in diameter) for more than one year5. Such superior stability and resistance to mechanical stress, have prompted the name gas “marble.” This robustness is attributed to cohesive forces generated from the triple liquid-solid-air interface30 and the use of glycerol (or SDS) as the liquid film to counter evaporation through the absorption of water molecules from the ambient air5. This unique gas encapsulation technology has wide-spread potential for use in the biomaterial, pharmaceutical, and cosmetic industries30 and supports the interest in entrapping medical gas or medical gas mixtures into gas marbles for the delivery of an MCM for cutaneous radiation injury.

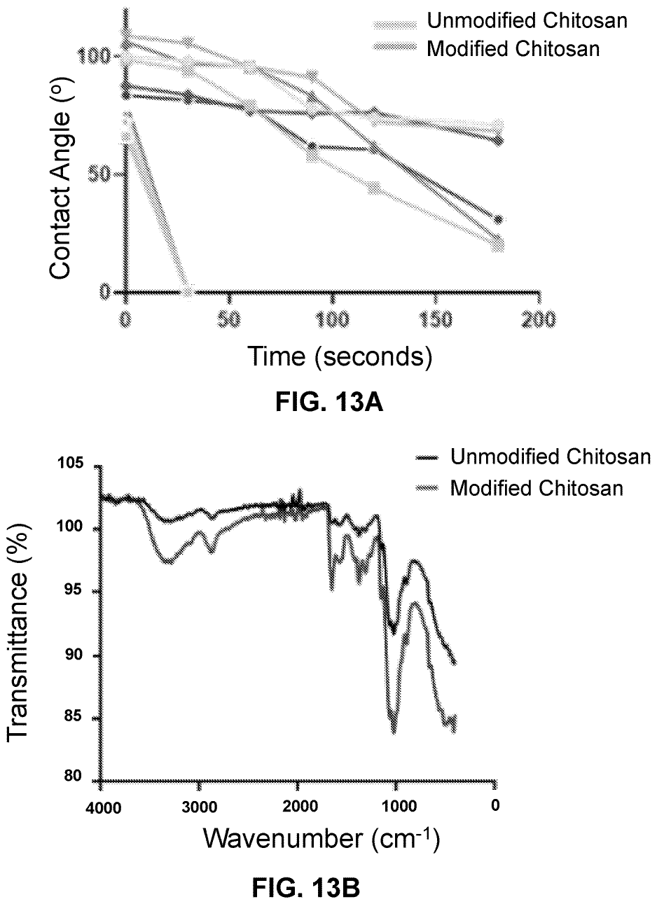

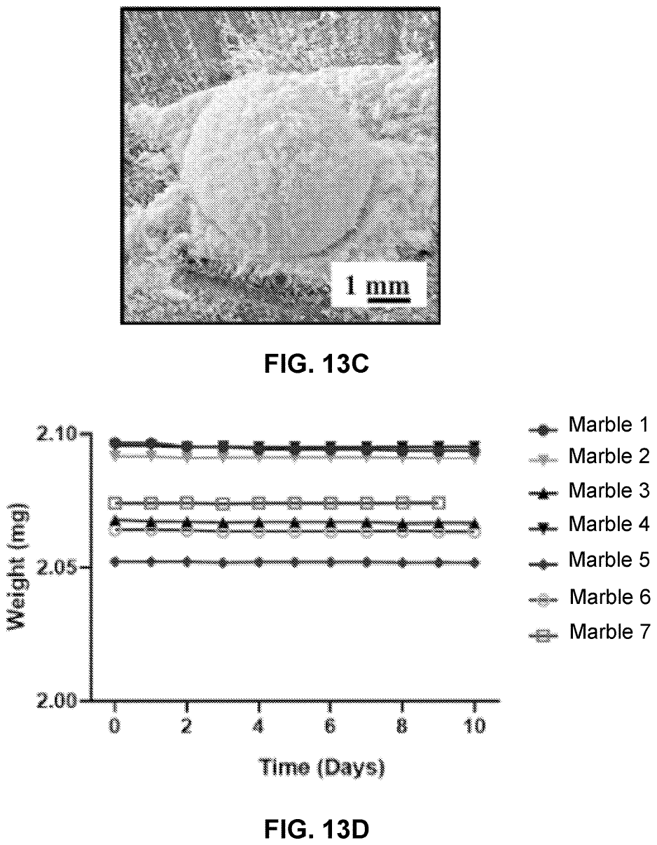

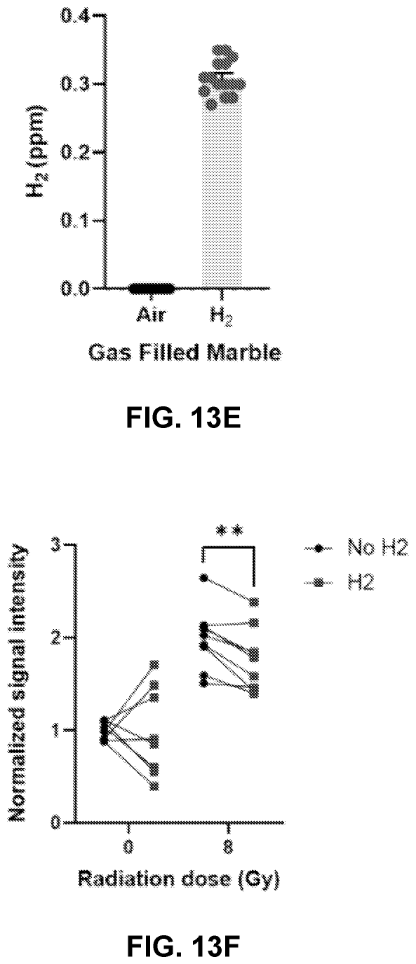

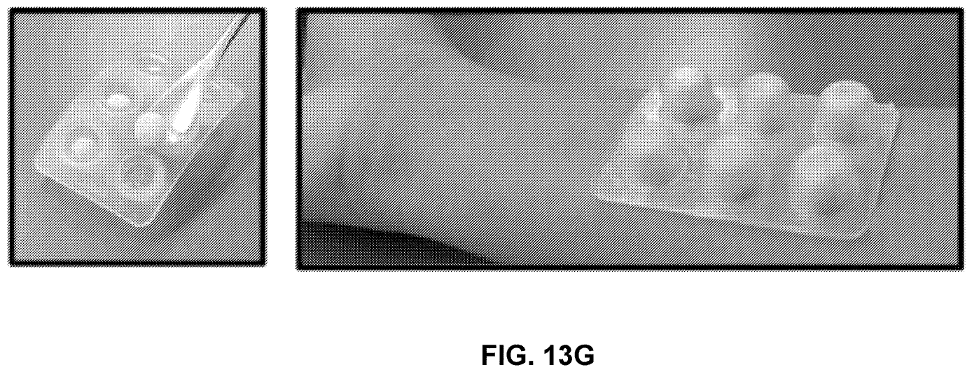

We explore the generation of biocompatible gas marbles which capture and deliver therapeutic gas for the treatment of cutaneous radiation injury. Using chitosan-derived microparticles, we demonstrated H₂-filled marble generation from a generally regarded as safe (GRAS) material. The particles were first modified by vapor treatment to increase their wettability (hydrophobicity)32 to improve the stability at the liquid-particle interface. Contact angle measurements showed a soft decrease compared to the unmodified control (FIG. 13A) which is supported by FTIR data indicating changes in intramolecular hydrogen (bands 2921, 2877) and C—H bonds (bands 3361, 3291) (FIG. 13B). We next tested the modified particles' ability to form marbles with H2 gas (FIG. 13C) and demonstrated they maintained their weight for 10 days, indicating good stability (FIG. 13D). Moreover, as the marble entrapped therapeutic gas, FIG. 13E showed the H2m concentration of fluid exposed to H2 gas-filled marbles yielded a dose of 0.31 ppm, indicating successful release of gas from the marble into the fluid. Interestingly, normal human dermal fibroblast cells irradiated by X-ray (8 Gy, 3 Gy/min) and exposed to 1.2 ppm H2 saturated cell culture medium showed decreased ROS levels (FIG. 13F). This data is supported by other in vitro studies where H2 was demonstrated to ameliorate cutaneous radiation injury by protecting cell viability, reducing ROS production, and lowering the apoptotic response, thus, suggesting that H2 gas is strong candidate for radiomitigation3,10,16,33-35. Taken together, this data supports use of gas marble technology for delivering radiomitigating therapeutic gas(es) as potential MCMs against cutaneous radiation injury.

A wide variety of gases are selected for testing according to their known role in reducing oxidative stress, inflammation, and apoptosis as well as the ability to promote wound healing and angiogenesis. Priority is given to CO, O2, and Xe and may also include NO, H2S, O3, Ar, and He. For the first time, these gases will be tested both individually and in combination with other gases, including with H2 to assess their ability to serve as a countermeasure to radiation. Accurate mixing (dosage) is achieved using our digital mass flow control system (Sierra Instruments) and sealed cell culture device.

For in vitro testing/screening, the cell culture medium is saturated with the appropriate dose of gas or gas mixture and exposed to 2D cultured dermal fibroblast cells (BJ normal human) as well as 3D full thickness skin equivalents (FTSE) (EpiSkin) post-irradiation. To saturate, medium will be degassed for 24 h under vacuum and then placed in a 1.8 L sealed chamber. The chamber is then flooded with the gas or mixture and held at atmospheric pressure for 24 h. After irradiation (0-10 Gy; 3 Gy/min), cultured cells and FTSE are exposed to gas-saturated medium at timepoints from 1 h to 48 h and assessed for radiomitigating effect. Cell viability is evaluated by Clonogenic survival and MTT assay or, for FTSE, by Live/Dead or TUNEL assay. Cellular oxidative stress will be investigated by measuring ROS levels. DNA double stranded break damage is assessed by γH2AX/53BP1 foci formation. Further, we determine lipid peroxidation (MDA) levels, glutathione/oxidized glutathione ratios, and 8-hydroxy-2-deoxy Guanosine (8-OHdG) levels. Collagen deposition is evaluated by Masson's trichrome staining. Also, specific to the FTSE, skin barrier function is quantitatively evaluated by transepithelial electrical resistance (TEER) measurements and permeation tests. Gaseous species producing positive radiomitigating effects in vitro are further assessed to assess DNA damage repair which is evaluated by the measurement of the expression level of proteins involved in homologous recombination (ATM, Rad51) and non-homologous end joining (Ku70, XRCC4, DNA-PKs) repair pathways by western blot.

Optimal gas dosing regimens are assessed, for example, H2: 1.0-1.6 ppm; Xe: 2.5×105-7.5×105 ppm; CO: 250-750 ppm.

Gaseous conditions demonstrating promising radiomitigating properties will then be encapsulated into gas marbles. Formulations specific to each gas and/or mixture will be optimized using biocompatible materials. Stability of marbles containing radiomitigating gas and/or mixtures are evaluated in an environmental chamber at 60% humidity (RH) over time by measuring mass and diameter. Additional testing by 129Xe NMR reveals gas exchange information, pore size, and ageing effects36.

Male SKH-1 mice (~10 weeks) are divided into 2 treatment groups: Control (air-filled gas marble) and treatment (H2-filled gas marble) with 18 animals/group. Based on preliminary results, additional gaseous treatment groups can be added. Prior to radiation exposure, rodent dorsal skin to be irradiated is marked and photographed. Then, under anesthesia, a custom clamping device37 isolates the dorsal tissue while protecting the body with lead shielding. Isolated skin will be irradiated at 45 Gy (X-RAD320, Precision X-Ray, 320 kV, 12.5 mA) to induce cutaneous radiation injury. 24 h post-irradiation, mice will begin radiomitigating gas marble treatment. A custom designed “marble patch” with chambers loaded with gas marbles (FIG. 13G) and adapted for small rodent size is topically applied to the site of injury and compressed to deliver ppm concentrations of H2 3×day. Gas delivery to tissues is measured using hydrogen microsensor (Unisense, Denmark). Mice are monitored for radiation toxicity, weight loss, changes in behavior, and skin damage.

Every week for 8 weeks, the skin is photographed for skin scoring assessment of wound healing (%) based on the Kumar scale and analyzed by ImageJ, where wound healing rate (%)=(original wound area−unhealed wound area)/original wound area38,39. Then, every other week, 3 mice are sacrificed for tissue collection from both irradiated (dorsal) and non-irradiated (ventral) sites. Tissues are analyzed by H&E staining, to assess dermal thickening as well as by Masson's trichrome staining for collagen deposition, and by picrosirius staining for fibrotic markers. Moreover, immunofluorescent staining for apoptotic cells by TUNEL assay and the oxidative stress biomarker by 8-OHdG are performed. In addition, tissues are harvested to assess lipid peroxidation (MDA) and SOD enzyme levels by commercially available kits (thiobarbituric method and hydroxylamine method, respectively). In addition, blood is collected at sacrifice for assessment of inflammatory markers (IL-6, EGF, IL-1β) in the serum as assessed by ELISA. Furthermore, samples will undergo cutaneous rigidity (Young's modulus) testing by tensiometer (Commercial Scale, Co.). Potential alternative strategies to assessment could employ dermal smart stickers (DermTech STRATUM) for non-invasive epidermal tissue sampling, capable of generating samples for RNA and proteomic analysis while simultaneously reducing the number of animals.

This example identifies novel therapeutic gases with potent radiomitigating properties and establishes optimal dosage regimens for their effective use in mitigating radiation-induced damage. Additionally, the gas marble technology is used to deliver therapeutic gas both in vitro and in vivo and thus provides important insight regarding feasibility and optimization of the marble formulation. This provides for the development of new strategies for radiomitigation and enhanced preparedness against radiological emergencies. Refinement in automating the fabrication of gas marbles and using various shell nanomaterials, such as solid lipids nanoparticles, opens the capability of producing larger batches and functionalizing the gas marbles for more precise tissue targeting. Other tissue delivery modalities can be used, for example, by formulating gas marbles into enteric coated capsule for gastrointestinal applications.

### References for Example 6:

(1) DiCarlo, A. L.; Bandremer, A. C.; Hollingsworth, B. A.; Kasim, S.; Laniyonu, A.; Todd, N. F.; Wang, S.-J.; Wertheimer, E. R.; Rios, C. I. Cutaneous Radiation Injuries: Models, Assessment and Treatments. Radiat. Res. 2020, 194 (3), 315-344. https://doi.org/10.1667/RADE-20-00120.1.
(2) Nakao, A.; Sugimoto, R.; Billiar, T. R.; McCurry, K. R. Therapeutic Antioxidant Medical Gas. J. Clin. Biochem. Nutr. 2009, 44 (1), 1-13. https://doi.org/10.3164/jcbn.08-193R.
(3) Hirano, S.; Ichikawa, Y.; Sato, B.; Yamamoto, H.; Takefuji, Y.; Satoh, F. Molecular Hydrogen as a Potential Clinically Applicable Radioprotective Agent. Int. J. Mol. Sci. 2021, 22 (9), 4566. https://doi.org/10.3390/ijms22094566.
(4) Timounay, Y.; Pitois, O.; Rouyer, F. Gas Marbles: Much Stronger than Liquid Marbles. Phys. Rev. Lett. 2017, 118 (22), 228001. https://doi.org/10.1103/PhysRevLett.118.228001.
(5) Roux, A.; Duchesne, A.; Baudoin, M. Everlasting Bubbles and Liquid Films Resisting Drainage, Evaporation, and Nuclei-Induced Bursting. Phys. Rev. Fluids 2022, 7 (1), L011601. https://doi.org/10.1103/PhysRevFluids.7.L011601.
(6) Jelveh, S.; Kaspler, P.; Bhogal, N.; Mahmood, J.; Lindsay, P. E.; Okunieff, P.; Doctrow, S. R.; Bristow, R. G.; Hill, R. P. Investigations of Antioxidant-Mediated Protection and Mitigation of Radiation-Induced DNA Damage and Lipid Peroxidation in Murine Skin. Int. J. Radiat. Biol. 2013, 89 (8), 618-627. https://doi.org/10.3109/09553002.2013.782450.
(7) Xue, J.; Yu, C.; Sheng, W.; Zhu, W.; Luo, J.; Zhang, Q.; Yang, H.; Cao, H.; Wang, W.; Zhou, J.; Wu, J.; Cao, P.; Chen, M.; Ding, W.-Q.; Cao, J.; Zhang, S. The Nrf2/GCH1/BH4 Axis Ameliorates Radiation-Induced Skin Injury by Modulating the ROS Cascade. J. Invest. Dermatol. 2017, 137 (10), 2059-2068. https://doi.org/10.1016/j.jid.2017.05.019.
(8) Ryan, J. L. Ionizing Radiation: The Good, the Bad, and the Ugly. J. Invest. Dermatol. 2012, 132 (3, Part 2), 985-993. https://doi.org/10.1038/jid.2011.411.
(9) Ding, J.: Xu, K.; Xu, H.; Ji, J.; Qian, Y.; Shen, J. Advances in Gas Therapeutics for Wound Healing: Mechanisms, Delivery Materials, and Prospects. Small Struct. 2024, 5 (1), 2300151. https://doi.org/10.1002/sstr.202300151.
(10) Qiu, X.; Dong, K.; Guan, J.; He, J. Hydrogen Attenuates Radiation-Induced Intestinal Damage by Reducing Oxidative Stress and Inflammatory Response. Int. Immunopharmacol. 2020, 84, 106517. https://doi.org/10.1016/j.intimp.2020.106517.
(11) Htun, Y.; Nakamura, S.; Kusaka, T. Hydrogen and Therapeutic Gases for Neonatal Hypoxic-Ischemic Encephalopathy: Potential Neuroprotective Adjuncts in Translational Research. Pediatr. Res. 2021, 89 (4), 753-759. https://doi.org/10.1038/s41390-020-0998-z.
(12) Sano, M.; Suzuki, M.; Homma, K.; Hayashida, K.; Tamura, T.; Matsuoka, T.; Katsumata, Y.; Onuki, S.; Sasaki, J. Promising Novel Therapy with Hydrogen Gas for Emergency and Critical Care Medicine. Acute Med. Surg. 2017, 5 (2), 113-118. https://doi.org/10.1002/ams2.320.
(13) Radiobiology Textbook; Baatout, S., Ed.; Springer International Publishing: Cham, 2023. https://doi.org/10.1007/978-3-031-18810-7.
(14) Zhou, Y.; Sheng, X.; Deng, F.; Wang, H.; Shen, L.; Zeng, Y.; Ni, Q.; Zhan, S.; Zhou, X. Radiation-Induced Muscle Fibrosis Rat Model: Establishment and Valuation. Radiat. Oncol. 2018, 13 (1), 160. https://doi.org/10.1186/s13014-018-1104-0.
(15) Watanabe, S.; Fujita, M.; Ishihara, M.; Tachibana, S.; Yamamoto, Y.; Kaji, T.; Kawauchi, T.; Kanatani, Y. Protective Effect of Inhalation of Hydrogen Gas on Radiation-Induced Dermatitis and Skin Injury in Rats. J. Radiat. Res. (Tokyo) 2014, 55 (6), 1107-1113. https://doi.org/10.1093/jrr/rru067.
(16) Qian, L.; Li, B.; Cao, F.; Huang, Y.; Liu, S.; Cai, J.; Gao, F. Hydrogen-Rich PBS Protects Cultured Human Cells from Ionizing Radiation-Induced Cellular Damage. Nucl. Technol. Radiat. Prot. 2010, 25(1), 23-29.
(17) Witte, M. B.; Barbul, A. Role of Nitric Oxide in Wound Repair. Am. J. Surg. 2002, 183 (4), 406-412. https://doi.org/10.1016/S0002-9610(02)00815-2.
(18) Luo, J.; Chen, A. F. Nitric Oxide: A Newly Discovered Function on Wound Healing. Acta Pharmacol. Sin. 2005, 26 (3), 259-264. https://doi.org/10.1111/j.1745-7254.2005.00058.x.
(19) Motterlini, R.; Otterbein, L. E. The Therapeutic Potential of Carbon Monoxide. Nat. Rev. Drug Discov. 2010, 9(9 ), 728-743. https://doi.org/10.1038/nrd 3228.
(20) Byrne, J. D.; Gallo, D.; Boyce, H.; Becker, S. L.; Kezar, K. M.; Cotoia, A. T.; Feig, V. R.; Lopes, A.; Csizmadia, E.; Longhi, M. S.; Lee, J. S.; Kim, H.; Wentworth, A. J.; Shankar, S.; Lee, G. R.; Bi, J.; Witt, E.; Ishida, K.; Hayward, A.; Kuosmanen, J. L. P.; Jenkins, J.; Wainer, J.; Aragon, A.; Wong, K.; Steiger, C.; Jeck, W. R.; Bosch, D. E.; Coleman, M. C.; Spitz, D. R.; Tift, M.; Langer, R.; Otterbein, L. E.; Traverso, G. Delivery of Therapeutic Carbon Monoxide by Gas-Entrapping Materials. Sci. Transl. Med. 2022, 14 (651), eabl4135. https://doi.org/10.1126/scitranslmed.ab14135.
(21) Esencan, E.; Yuksel, S.; Tosun, Y. B.; Robinot, A.; Solaroglu, I.; Zhang, J. H. XENON in Medical Area: Emphasis on Neuroprotection in Hypoxia and Anesthesia. Med. Gas Res. 2013, 3 (1), 4. https://doi.org/10.1186/2045-9912-3-4.
(22) Fitzpatrick, E.; Holland, O. J.; Vanderlelie, J. J. Ozone Therapy for the Treatment of Chronic Wounds: A Systematic Review. Int. Wound J. 2018, 15 (4), 633-644. https://doi.org/10.1111/iwj.12907.
(23) Szabó, C. Hydrogen Sulphide and Its Therapeutic Potential. Nat. Rev. Drug Discov. 2007, 6 (11), 917-935. https://doi.org/10.1038/nrd2425.
(24) Wallace, J. L.; Wang, R. Hydrogen Sulfide-Based Therapeutics: Exploiting a Unique but Ubiquitous Gasotransmitter. Nat. Rev. Drug Discov. 2015, 14 (5), 329-345. https://doi.org/10.1038/nrd4433.
(25) Jin, R. C.; Loscalzo, J. Vascular Nitric Oxide: Formation and Function. J. Blood Med. 2010, 1, 147-162. https://doi.org/10.2147/JBM.S7000.
(26) Tai, S. Endothelial Nitric Oxide Synthase|Arteriosclerosis, Thrombosis, and Vascular Biology. https://www.ahajournals.org/doi/full/10.1161/01.ATV.0000109171.50229.33 (accessed 2024 Feb. 27).
(27) Nowrangi, D. S.; Tang, J.; Zhang, J. H. Argon Gas: A Potential Neuroprotectant and Promising Medical Therapy. Med. Gas Res. 2014, 4 (1), 3. https://doi.org/10.1186/2045-9912-4-3.
(28) Gong, W. Therapeutic gas delivery strategies-Gong-2022-WIREs Nanomedicine and Nanobiotechnology—Wiley Online Library. https://wires.onlinelibrary.wiley.com/doi/full/10.1002/wnan.1744 (accessed 2024 Mar. 1).
(29) Timounay, Y.; Ou, E.; Lorenceau, E.; Rouyer, F. Low Gas Permeability of Particulate Films Slows down the Aging of Gas Marbles. Soft Matter 2017, 13 (42), 7717-7720. https://doi.org/10.1039/C7SM01444A.
(30) Cervantes-Álvarez, A. M.; Escobar-Ortega, Y. Y.; Sauret, A.; Pacheco-Vázquez, F. Air Entrainment and Granular Bubbles Generated by a Jet of Grains Entering Water. J. Colloid Interface Sci. 2020, 574, 285-292. https://doi.org/10.1016/j.jcis.2020.04.009.
(31) Harris, A. F.; Lacombe, J.; Zenhausern, F. The Emerging Role of Decellularized Plant-Based Scaffolds as a New Biomaterial. Int. J. Mol. Sci. 2021, 22 (22), 12347. https://doi.org/10.3390/ijms222212347.
(32) Kurita, K.; Hirakawa, M.; Kikuchi, S.; Yamanaka, H.; Yang, J. Trimethylsilylation of Chitosan and Some Properties of the Product. Carbohydr. Polym. 2004, 56 (3), 333-337. https://doi.org/10.1016/j.carbpol.2004.02.008.
(33) Terasaki, Y.; Ohsawa, I.; Terasaki, M.; Takahashi, M.; Kunugi, S.; Dedong, K.; Urushiyama, H.; Amenomori, S.; Kaneko-Togashi, M.; Kuwahara, N.; Ishikawa, A.; Kamimura, N.; Ohta, S.; Fukuda, Y. Hydrogen Therapy Attenuates Irradiation-Induced Lung Damage by Reducing Oxidative Stress. Am. J. Physiol.-Lung Cell. Mol. Physiol. 2011, 301 (4), L415-L426. https://doi.org/10.1152/ajplung.00008.2011.
(34) Qian, L.; Cao, F.; Cui, J.; Huang, Y.; Zhou, X.; Liu, S.; Cai, J. Radioprotective Effect of Hydrogen in Cultured Cells and Mice. Free Radic. Res. 2010, 44 (3), 275-282. https://doi.org/10.3109/10715760903468758.
(35) Kawasaki, H.; Guan, J.; Tamama, K. Hydrogen Gas Treatment Prolongs Replicative Lifespan of Bone Marrow Multipotential Stromal Cells in Vitro While Preserving Differentiation and Paracrine Potentials. Biochem. Biophys. Res. Commun. 2010, 397 (3), 608-613. https://doi.org/10.1016/j.bbrc.2010.06.009.
(36) Wisser, D.; Hartmann, M. 129Xe NMR on Porous Materials: Basic Principles and Recent Applications. Adv. Mater. Interfaces 2021, 8 (4), 2001266. https://doi.org/10.1002/admi.202001266.
(37) Rifkin, L. H.; Stojadinovic, S.; Stewart, C. H.; Song, K. H.; Maxted, M. C.; Bell, M. H.; Kashefi, N. S.; Speiser, M. P.; Saint-Cyr, M.; Story, M. D.; Rohrich, R. J.; Brown, S. A.; Solberg, T. D. An Athymic Rat Model of Cutaneous Radiation Injury Designed to Study Human Tissue-Based Wound Therapy. Radiat. Oncol. 2012, 7 (1), 68. https://doi.org/10.1186/1748-717X-7-68.
(38) Kumar, S.; Kolozsvary, A.; Kohl, R.; Lu, M.; Brown, S.; Kim, J. H. Radiation-Induced Skin Injury in the Animal Model of Scleroderma: Implications for Post-Radiotherapy Fibrosis. Radiat. Oncol. 2008, 3 (1),40. https://doi.org/10.1186/1748-717X-3-40.
(39) Mei, K.; Zhao, S.; Qian, L.; Li, B.; Ni, J.; Cai, J. Hydrogen Protects Rats from Dermatitis Caused by Local Radiation. J. Dermatol. Treat. 2014, 25 (2), 182-188. https://doi.org/10.3109/09546634.2012.762639

### Example 7: Gas Marble Therapeutics for the Management of Drug-Resistant Melanoma

Of the skin cancers, melanoma remains the most aggressive and therapy-resistant form^{1,2}. Once this disease becomes metastatic, the prognosis is very poor, accounting for 75% of skin cancer deaths^{3,4}. Recently, genetic advancements and an improved understanding of melanoma's pathogenesis have given way to new therapeutic modalities. Despite significant progress in clinical management, issues with low patient response rates and resistance to targeted therapies persist⁵. To overcome these challenges, novel treatments are needed.

Medical gas therapy in the form of gasotransmitters (e.g., CO, NO, H2S), noble gases (e.g., He, Xe), or traditional gases (e.g., O₂, O₃) has received increasing attention as a therapeutic approach in cancer treatment⁶⁻⁸. Many of these gases have shown the ability to decrease tumor volume, reduce cell survival, and induce apoptosis while simultaneously protecting normal tissues and evading the onset of drug resistance commonly associated with conventional therapies^{6,9-11}. Despite these results, gaseous species, either alone or in combination, have not yet been thoroughly investigated as treatments for melanoma. This is due to gas delivery being limited to inhalation or gas-releasing molecules, both of which suffer from issues with precise delivery, leading to inaccurate dosing. Thus, a new, stable medical gas delivery system is needed.

Recently, it has been shown that liquid film-entrapped gas can be fortified with micro/nanoparticles to generate “gas marbles” which are stable for over one year^{12,13}. With two liquid-gas interfaces providing superior mechanical strength, these gas-containing marbles can oppose escaping gas at pressures up to 10 times the Laplace pressure. Clinically, these highly resistive marble formulations can be used as a controlled delivery vehicle in which to entrap therapeutic gas(es) for the treatment of drug-resistant melanoma.

To screen therapeutic gases for their ability to augment the efficacy of melanoma therapy against drug-resistant phenotypes: Using drug-resistant melanoma cells in vitro, medical gases and medical gas combinations are explored for their ability to enhance therapeutic outcomes in drug-resistant melanoma when used alone and alongside conventional targeted therapy. Parameters such as cellular viability, migration, invasiveness, melanocytic/undifferentiated phenotypes, and cell death pathways are evaluated.

To establish a method to assess the gas dosage delivered by the gas marble delivery system using analytical technique(s): To investigate the critical dosage parameter, various analytical approaches (e.g., gas chromatography) are explored for their ability to verify the gas dose within the marble delivery system and quantify the therapeutic gas concentration (ppm).

This example is the foundation for use of medical gases to sensitize drug-resistant melanoma to traditional therapy. In addition, we develop a reproducible method by which the pharmaceutical gas dose within the gas marble can be quantitatively assessed. This work contributes toward the delivery of therapeutic gas by gas marbles packaged for oral delivery to improve outcomes for melanoma patients. The data serves in applications related to gas marble delivery studies in murine models as well as new therapeutic modalities for managing melanoma malignancies.

Gaseous molecules have been increasingly explored for therapeutic development as medical gas therapy has been shown to act alone and synergistically with cancer therapies to enhance the effect of traditional treatments, reducing toxic side effects, and even protecting normal cells⁸. In particular, the gasotransmitter hydrogen sulfide (H₂S) is endogenously produced to regulate mitochondrial bioenergetics¹⁴. At doses higher than endogenic levels (~3 ppm), H₂S-donor compounds have been shown to exhibit anticancerous effects in vitro and in vivo, inhibiting melanoma cell proliferation by suppressing the expression of NFκB, PI3/AKT, and other anti-apoptotic proteins while not affecting the viability of normal cells^{9,15}. In murine models, topical delivery of another signal transduction molecule, carbon monoxide (CO), by CO-releasing molecules (CORMS) (250-500 μM) has been shown to increase the regression of established cutaneous tumors and inhibit the development of malignant and locally invasive tumors¹⁶-18. Synergistically, CO-releasing nanomaterials used in combination with the chemotherapy drug doxorubicin showed significant cytotoxicity in vivo against solid prostate tumors, suggesting CO can enhance antitumor efficiency while reducing side effects for patients. Other pharmaceutical gases (e.g., H₂, O₃, O₂, Xe) have demonstrated efficacy in modulating tumor volume, decreasing cell survival, alleviating immune suppression in the tumor microenvironment, and promoting cell death pathways^{8,19-23}. Each delivers such anticancer effects without negatively affecting healthy tissues, inducing harmful side effects, or promoting drug resistance.

Favorably, many of these gases, are widely available for a low cost and have received prior FDA approval for healthcare indications. Their use can circumvent the high expense and long development times associated with new pharmaceutical candidates²⁴. However, translation has largely been limited due to the challenge of gas delivery. Traditional delivery methods provide the gas for patients via inhalation or by gas-releasing platforms or prodrugs. Unfortunately, both approaches result in uncontrolled and inaccurate dosing²⁵.

Recent studies have explored the use of gas marble technology where gas is entrapped by a liquid film (e.g., a bubble)^{12,13}. Normally, at atmospheric pressure, gravity-induced drainage would cause this bubble to spontaneously burst and release the entrapped gas. However, by adding partially wetted particles to the air-liquid interface, the bubble becomes fortified and able to maintain its integrity (without change in diameter) for more than one year¹³. Such superior stability and resistance to mechanical stress, have prompted the name gas “marble.” This robustness is attributed to cohesive forces generated from the triple liquid-solid-air interface and the use of glycerol (or SDS) as the liquid film to counter evaporation through the absorption of water molecules from the ambient air¹³. This unique gas encapsulation technology has wide-spread potential for use in the biomaterial, pharmaceutical, and cosmetic industries and supports the interest in the instant example to entrap medical gas or medical gas mixtures into gas marbles for the co-delivery of a traditional drug (e.g. Vemurafenib, Dabrafenib) with therapeutic gas for the treatment of melanoma.

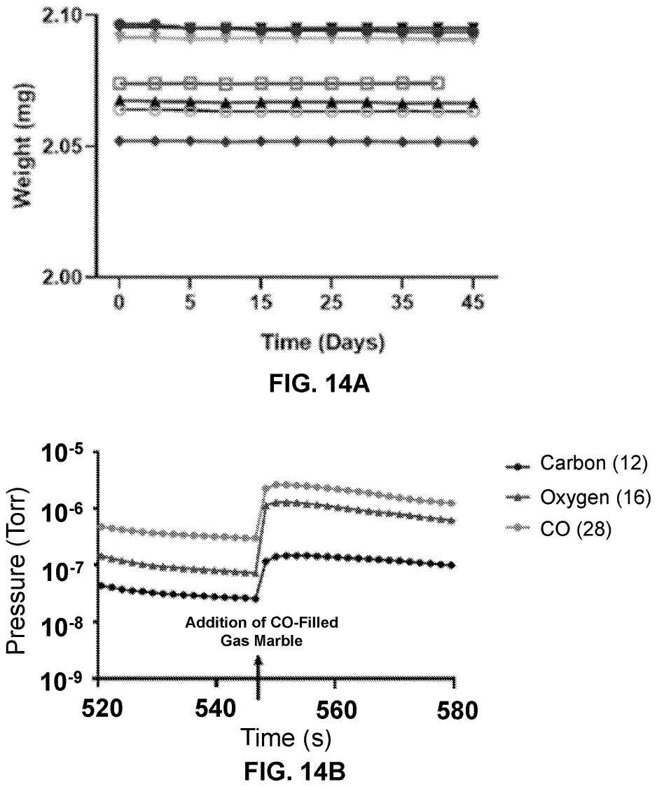

As such, our expertise with naturally derived biomaterials recently led us to explore the generation of biocompatible gas marbles that capture and deliver therapeutic gas for the treatment of melanoma. Using chitosan-derived microparticles, we demonstrate CO-filled marble generation from a generally regarded as safe (GRAS) material. The particles are first modified by vapor treatment to increase their wettability (hydrophobicity) to improve the stability at the liquid-particle interface. We test the modified particles' ability to form marbles with CO gas (FIG. 13C) and demonstrated they maintained their weight for 45 days, suggesting good stability (FIG. 13D). Moreover, FIG. 14 showed data collected by Residual Gas Analysis. Upon the addition of the CO-filled gas marble (indicated by arrow), a heightened peak intensity for carbon, oxygen, and CO is observed, indicating the presence of CO gas within the marble.

This gas entrapment data is supported by in vivo and in vitro studies where gaseous molecules are demonstrated to ameliorate melanoma tumor burden by increasing the apoptotic response while concurrently protecting normal tissues thus, suggesting that medical gases are strong candidates to aid in overcoming therapy resistance in metastatic melanoma. This reflects the promise of gas marble technology for the delivery of medical gas therapy to sensitize drug-resistant melanoma to traditional treatment modalities.

Aims for this example include: (1) Screening therapeutic gases for their ability to augment the efficacy of melanoma therapy against drug-resistant phenotypes; and (2) Establishing a method to assess the gas dosage delivered by the gas marble delivery system using analytical technique(s).

Gaseous species, such as CO, H₂S, and H₂, are screened for their ability to sensitize drug-resistant melanoma to traditional therapy. Gas screening mixture groups may also include O₃, O₂, Xe, and Ar. For the first time, these gases are tested both individually and in combination, for their capacity to treat drug-resistant melanoma.

For in vitro testing/screening, human malignant melanoma cells (BRAF mutated SK-MEL-28, A-375) are first exposed to increasing concentrations of BRAF inhibitor (Dabrafenib, up to 1.5 μM) for five days to generate a drug-resistant subline²⁶. Then, accurate gas mixing (dosing) is achieved using our digital mass flow control system (Sierra Instruments) where the cell culture medium is saturated with therapeutic gas.

For melanoma treatment, ANOVA analysis compares the efficiency of different treatment groups: no treatment, gas only, BRAF inhibitor only, and gas+BRAF inhibitor. The “gas only” group is a control that also serves as an opportunity to test if the gas on its own can inhibit cancer cell growth. Treatment times from 1-3 days are tested. Optimal gas dosing regimens are assessed, for example, CO: 250-750 ppm. Normal human dermal fibroblast cells (BJ) are also used to assess potential gas toxicity. Cell viability is evaluated by MTT assay while the apoptosis pathway is investigated by TUNEL assay. Cell migration studies are performed by scratch assay. Invasiveness is assessed by migration assay using our transwell system. Gaseous species and their combinations producing antitumor effects in vitro are further assessed by qRT-PCR for melanocytic/undifferentiated phenotype.

Gaseous conditions demonstrating promising anticancer properties are encapsulated into gas marbles. The stability of marbles containing therapeutic gas and/or mixtures is evaluated in an environmental chamber at 60% humidity (RH) over time by measuring mass and diameter.

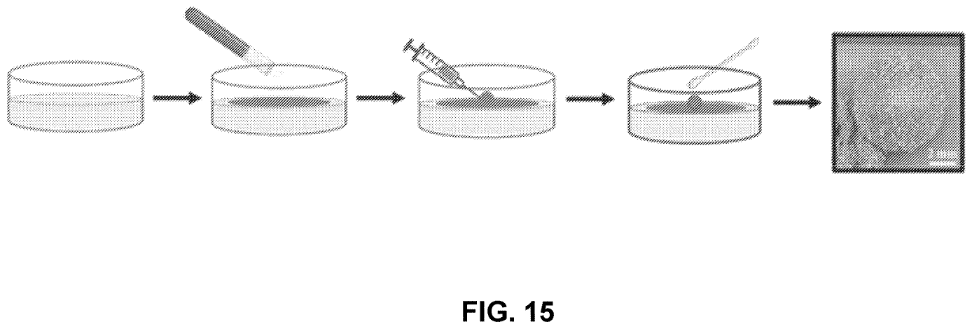

To establish a robust methodology, accessible analytical techniques are evaluated for their ability to quantify gas marble dosages. Examples include, but are not limited to, Gas Chromatography (GC), Residual Gas Analysis (RGA), Gas Chromatography with Mass Spectroscopy (GC/MS)²⁷. Carbon monoxide gas (500 ppm dose) is used to establish the procedure due to its low atmospheric presence (50-100 ppb)²⁸. To form therapeutic gas marbles for gas analysis, medical gases are accurately mixed using a digital mass flow control system (Sierra Instruments). Medical grade air containing 1000 ppm CO is mixed with standard medical grade air at a 1:1 ratio to achieve a dose of 500 ppm. Gas flows into an airtight sampling bag (Tedlar) and is withdrawn using a needling and airtight syringe (Hamilton) through a septum. Gas marbles are formed using the airtight syringe, where the medical gas is injected below a raft of chitosan particles floating on 70% glycerol (FIG. 15). The injected air forms a dome shape in the particle raft. The dome is rolled through the particle raft using a laboratory spatula to fortify the gas marble.

CO-dosed marbles are assessed using a Center's Waters GCT Premier Time of Flight mass spectrometer with an Agilent 7890 by dissolving the marble into the solvent and loading it into the instrument by SPME liquid-injection system (LEAP Technologies). The instrument is adapted with a flame ionization detector (FID)²⁹ with a high concentration of nitrogen. Propak Q chromatographic columns are used to separate nitrogen from the detection system while CO is in-line separated and converted to methane (CH₄) using a nickel catalyst to increase sensitivity detection. The resulting chromatograms show CO appearing as a single peak with a characteristic tR. Linearities are expected to detect CO in the range of sub 5 ppm levels with accuracy and reproducibility (RSD<2%). Air-filled marbles serve as the experimental control while high-purity CO gas (Agilent) serve as the reference material. Optimization of the sample preparation, detector response, temperature programming, carrier gas flow rate, signal-to-noise ratio, and baseline stability is performed to enhance instrument sensitivity and peak resolution.

Alternatively, an RGA (SRS200), where marble samples can be agitated under heat and rough vacuum, can be used to release the entrapped CO into the instrument inlet. Molecules are ionized by a high-voltage hot filament, accelerated into a mass separation filter, and detected by the RGA head with a quadrupole probe. Results are obtained based on a mass spectrum of partial pressure vs. gas species mass. Detection limits are 10⁸ ppm range at 10⁻⁵⁻¹¹ Torr. As needed, sensitivity is increased using the electron multiplier to partial pressures down to 5⁻¹⁴ Torr.

### References for Example 7:

1. Eggermont, A. M., Spatz, A. & Robert, C. Cutaneous melanoma. The Lancet 383, 816-827 (2014).
2. Schadendorf, D. et al. Melanoma. Nat. Rev. Dis. Primer 392, 971-984 (2018).
3. Domingues, B., Lopes, J. M., Soares, P. & Pópulo, H. Melanoma treatment in review. ImmunoTargets Ther. 7, 35-49 (2018).
4. Davis, L. E., Shalin, S. C. & Tackett, A. J. Current state of melanoma diagnosis and treatment. Cancer Biol. Ther. 20, 1366-1379 (2019).
5. Guo, W., Wang, H. & Li, C. Signal pathways of melanoma and targeted therapy|Signal Transduction and Targeted Therapy. https://www.nature.com/articles/s41392-021-00827-6?fromPaywallRec=false (2021).
6. Ji, P. et al. Mechanisms and Application of Gas-Based Anticancer Therapies. Pharmaceuticals 16, 1394 (2023).
7. Li, S. et al. Hydrogen Gas in Cancer Treatment. Front. Oncol. 9, (2019).
8. Jing, Y.-Z., Li, S.-J. & Sun, Z.-J. Gas and gas-generating nanoplatforms in cancer therapy. J. Mater. Chem. B 9, 8541-8557 (2021).
9. De Cicco, P. et al. The Hydrogen Sulfide Releasing Molecule Acetyl Deacylasadisulfide Inhibits Metastatic Melanoma. Front. Pharmacol. 8, (2017).
10. Wu, M. et al. Recent advances in the development of nitric oxide-releasing biomaterials and their application potentials in chronic wound healing. J. Mater. Chem. B 9, 7063-7075 (2021).
11. Huerta, S. Nitric oxide for cancer therapy. Future Sci. OA 1, FSO44 (2015).
12. Timounay, Y., Pitois, O. & Rouyer, F. Gas Marbles: Much Stronger than Liquid Marbles. Phys. Rev. Lett. 118, 228001 (2017).
13. Roux, A., Duchesne, A. & Baudoin, M. Everlasting bubbles and liquid films resisting drainage, evaporation, and nuclei-induced bursting. Phys. Rev. Fluids 7, L011601 (2022).
14. Szabo, C. et al. Regulation of mitochondrial bioenergetic function by hydrogen sulfide. Part I. Biochemical and physiological mechanisms. Br. J. Pharmacol. 171, 2099-2122 (2014).
15. Hellmich, M. R., Coletta, C., Chao, C. & Szabo, C. The Therapeutic Potential of Cystathionine β-Synthetase/Hydrogen Sulfide Inhibition in Cancer. Antioxid. Redox Signal. 22, 424-448 (2015).
16. Allanson, M. & Reeve, V. E. Carbon monoxide signalling reduces photocarcinogenesis in the hairless mouse. Cancer Immunol. Immunother. 56, 1807-1815 (2007).
17. Tien Vo, T. T. et al. The potentials of carbon monoxide-releasing molecules in cancer treatment: An outlook from ROS biology and medicine. Redox Biol. 46, 102124 (2021).
18. Motterlini, R. & Otterbein, L. E. The therapeutic potential of carbon monoxide. Nat. Rev. Drug Discov. 9, 728-743 (2010).
19. Wang, B. & Otterbein, L. E. Endogenous CO Production in Sickness and in Health. in Carbon Monoxide in Drug Discovery 1-26 (John Wiley & Sons, Ltd, 2022). doi:10.1002/9781119783435.ch1.
20. Ohta, S. Recent Progress Toward Hydrogen Medicine: Potential of Molecular Hydrogen for Preventive and Therapeutic Applications. Curr. Pharm. Des. 17, 2241-2252 (2011).
21. Wang, B. et al. Hydrogen: A Novel Treatment Strategy in Kidney Disease. Kidney Dis. 8, 126-136 (2022).
22. Matei, N., Camara, R. & Zhang, J. H. Emerging mechanisms and novel applications of hydrogen gas therapy. Med. Gas Res. 8, 98-102 (2018).
23. Büttner, T. et al. Combining a noble gas with radiotherapy: glutamate receptor antagonist xenon may act as a radiosensitizer in glioblastoma. Radiat. Oncol. 19, 16 (2024).
24. Nowrangi, D. S., Tang, J. & Zhang, J. H. Argon gas: a potential neuroprotectant and promising medical therapy. Med. Gas Res. 4, 3 (2014).
25. Nakao, A., Sugimoto, R., Billiar, T. R. & McCurry, K. R. J. Clin. Biochem. Nutr. 44, 1-13 (2009).
26. Caporali, S. et al. Targeting the pi3k/akt/mtor pathway overcomes the stimulating effect of dabrafenib on the invasive behavior of melanoma cells with acquired resistance to the braf inhibitor. Int. J. Oncol. 49, 1164-1174 (2016).
27. Klegerman, M. E. et al. Gas chromatography/mass spectrometry measurement of xenon in gas-loaded liposomes for neuroprotective applications. Rapid Commun. Mass Spectrom. 31, 1-8 (2017).
28. Carbon Monoxide|Center for Science Education. https://scied.ucar.edu/learning-zone/air-quality/carbon-monoxide.
29. Porter, K. & Volman, D. H. Flame Ionization Detection of Carbon Monoxide for Gas Chromatographic Analysis. Anal. Chem. 34, 748-749 (1962).

### Example 8: Biocompatible Gas Marbles

This example investigates biocompatible materials useful for pharmaceutical formulations. Biocompatible materials that the Food and Drug Administration (FDA) classifies as generally regarded as safe (GRAS) have been selected for investigation. Cellulose-based materials generally prove to be too hydrophilic and the resultant marbles overly unstable. Thus, we explore chitosan, a renewable amino polysaccharide widely used in the food, cosmetic, and pharmaceutical industries. Using chitosan-derived microparticles, we demonstrate that biocompatible gas marbles can be generated and filled with medical gaseous species.

The chitosan microparticles were first modified by vapor treatment (4-6 h) using an organosilicon, hexamethyldisilizane (HMDS), to increase their wettability (hydrophobicity). Such treatment improves the stability at the liquid-solid (particle) interface. To assess the effects of the treatment, Fourier-transform infrared spectroscopy (FTIR) showed changes in wavelength at 2921 and 2877, suggesting the modification of intramolecular hydrogen bonds. In addition, the modified bands 3361 and 3291 suggest the conversion of C—H stretching while variation at band 1645 is indicative of the acetyl group modification. Next, we performed contact angle measurements using a goniometer which showed the modified chitosan particles to have an average contact angle of 106.46° with a soft decrease over 150 seconds, compared to the unmodified control which exhibited a 71.57° average contact angle sharp decrease in less than 50 seconds. This suggests a successful HMDS modification, a quenching of the hydroxyl groups, leading to the increase in hydrophobicity. We next successfully tested the modified particles' ability to reproducibly form gas marbles with H₂ gas. The weight of these H₂-filled marbles were examined over time and found that the weight is maintained for 100 days, reflecting good stability. Moreover, these biocompatible marbles entrapped therapeutic gas. FIG. 13E shows the H₂ concentration of fluid exposed to H₂ gas-filled marbles yielded a dose of 0.31 ppm, indicating the successful release of gas from the marble into the fluid.

We developed a scalable skin patch that can be used to load and deliver therapeutic gas marbles of various sizes in vivo. In FIG. 13G, the patch is loaded with H₂-filled gas marbles and secured to human skin. This is but one example of applying a gas marble to a biological system (in this example human skin)

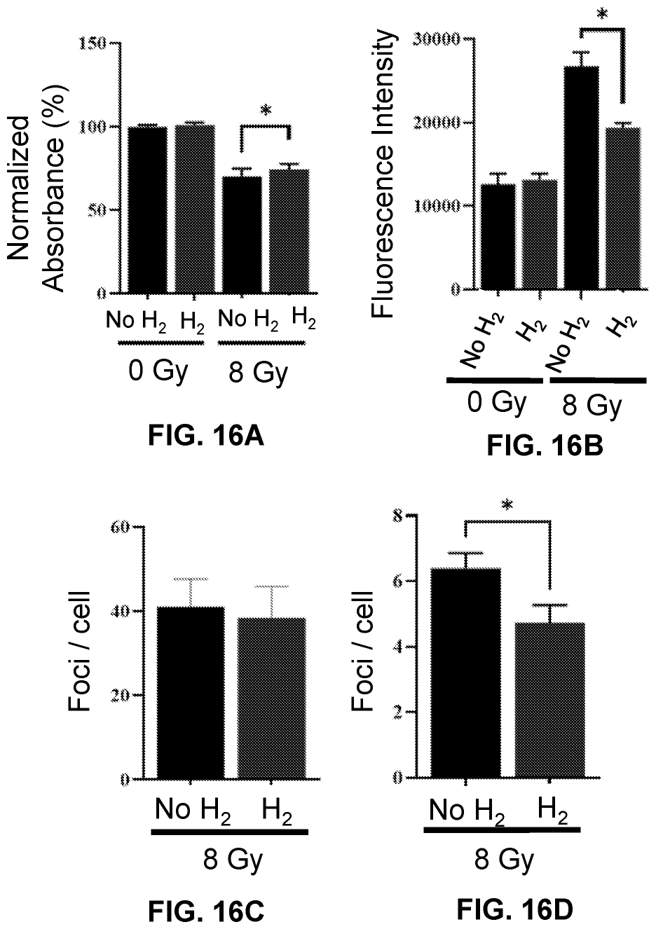

In addition, we use a robust protocol for in vitro testing to screen gaseous species for their potential as therapeutic agents. Hydrogen gas is a strong antioxidant, neutralizer of free radicals, and protectant against oxidative stress. With its small size, H₂ can easily diffuse across the cell membrane and act as a powerful signaling molecule. Thus, our next steps explored the potential of H₂ gas as a radiomitigator against cutaneous radiation injury. Normal human dermal fibroblast cells were irradiated by X-ray (8 Gy, 3 Gy/min) and exposed to 1.2 ppm H₂ saturated cell culture medium. Results showed that irradiated fibroblast cells exposed to H₂ treatment had an increase in normalized absorbance, suggesting improved cell viability (FIG. 16A). In addition, the fluorescence intensity of reactive oxygen species (ROS) was found to be lower in the irradiated cells in the presence of hydrogen gas, compared to the non-treated control (FIG. 16B), suggesting a reduction of free radicals. Finally, treatment with molecular hydrogen reduced the number of DNA double-stranded break repair proteins 24 hours post-irradiation exposure. These damaging double-stranded breaks occur when the ionizing radiation directly cuts through the DNA and also generates sustained oxidative stress (ROS) which indirectly damages the DNA. In immunofluorescent stained images (not shown), the cell nucleus is in blue and the double-stranded break repair protein (gamma histone (γH2AX)) is stained red/pink. The repair process peaks one-hour post-irradiation exposure (FIG. 16C) and returns to baseline levels 24-h post-IR where the treated group has fewer repair sites (FIG. 16D). Gas screening and testing investigations are ongoing with molecular hydrogen, carbon monoxide, and noble gases (Ar, Xe), each alone and in combination to assess their ability to produce a therapeutic effect.

### Statements Regarding Incorporation by Reference and Variations

All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, exemplary embodiments and optional features, modification and variation of the concepts herein disclosed may be resorted to by those skilled in the art, and that such modifications and variations are considered to be within the scope of this invention as defined by the appended claims. The specific embodiments provided herein are examples of useful embodiments of the present invention and it will be apparent to one skilled in the art that the present invention may be carried out using a large number of variations of the devices, device components, methods steps set forth in the present description. As will be obvious to one of skill in the art, methods and devices useful for the present methods can include a large number of optional composition and processing elements and steps.

When a group of substituents is disclosed herein, it is understood that all individual members of that group and all subgroups, including any isomers, enantiomers, and diastereomers of the group members, are disclosed separately. When a Markush group or other grouping is used herein, all individual members of the group and all combinations and subcombinations possible of the group are intended to be individually included in the disclosure. When a compound is described herein such that a particular isomer, enantiomer or diastereomer of the compound is not specified, for example, in a formula or in a chemical name, that description is intended to include each isomers and enantiomer of the compound described individual or in any combination. Additionally, unless otherwise specified, all isotopic variants of compounds disclosed herein are intended to be encompassed by the disclosure. For example, it will be understood that any one or more hydrogens in a molecule disclosed can be replaced with deuterium or tritium. Isotopic variants of a molecule are generally useful as standards in assays for the molecule and in chemical and biological research related to the molecule or its use. Methods for making such isotopic variants are known in the art. Specific names of compounds are intended to be exemplary, as it is known that one of ordinary skill in the art can name the same compounds differently.

Many of the molecules disclosed herein contain one or more ionizable groups [groups from which a proton can be removed (e.g., —COOH) or added (e.g., amines) or which can be quaternized (e.g., amines)]. All possible ionic forms of such molecules and salts thereof are intended to be included individually in the disclosure herein. With regard to salts of the compounds herein, one of ordinary skill in the art can select from among a wide variety of available counterions those that are appropriate for preparation of salts of this invention for a given application. In specific applications, the selection of a given anion or cation for preparation of a salt may result in increased or decreased solubility of that salt.

Every formulation or combination of components described or exemplified herein can be used to practice the invention, unless otherwise stated.

Whenever a range is given in the specification, for example, a temperature range, a time range, or a composition or concentration range, all intermediate ranges and subranges, as well as all individual values included in the ranges given are intended to be included in the disclosure. It will be understood that any subranges or individual values in a range or subrange that are included in the description herein can be excluded from the claims herein.

All patents and publications mentioned in the specification are indicative of the levels of skill of those skilled in the art to which the invention pertains. References cited herein are incorporated by reference herein in their entirety to indicate the state of the art as of their publication or filing date and it is intended that this information can be employed herein, if needed, to exclude specific embodiments that are in the prior art. For example, when composition of matter are claimed, it should be understood that compounds known and available in the art prior to Applicant's invention, including compounds for which an enabling disclosure is provided in the references cited herein, are not intended to be included in the composition of matter claims herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms. The invention illustratively described herein suitably may be practiced in the absence of any element or elements, limitation or limitations which is not specifically disclosed herein.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

1. A method of treating a biological system with a gas marble, the method comprising the steps of:
providing a gas marble containing a gaseous chemical compound useful for treating the biological system, wherein the gaseous chemical compound is from a planetary source; and
applying the gas marble to the biological system;
thereby treating the biologic system with the gas marble.

2. The method of claim 1, wherein the step of providing the gas marble comprises the steps of:
identifying the gaseous chemical compound from the planetary source;
collecting the gaseous chemical compound from the planetary source;
providing a gas container in fluid communication with a liquid vessel that contains a fluid mixture having a selected physico-chemical property, wherein the fluid mixture is configured to support an assembly of composite nanomaterials;
loading the collected gaseous chemical compound into the gas container; and
activating the loaded gaseous chemical compound in the gas container with the assembly of composite nanomaterials, wherein the assembly of composite nanomaterials are partially wet, to generate bubbles in said fluid mixture and form a stable gas marble containing the gaseous chemical compound.

3. The method of claim 1, wherein the planetary source is Earth atmosphere, an atmosphere of a planet in the solar system, or from an exo-planet.

4. The method of claim 2, wherein the selected physico-chemical property comprises viscosity, resistance to evaporation, or a mechanical stress, wherein the physico-chemical property influences a fluid interface, including an gas-liquid interface.

5. The method of claim 2, further comprising the step of:
optimizing a physical gas parameter, such as a gas mass, concentration and/or a gas temperature, for computing a gas flow and a gas spatial variation within a planet climate condition to identify a planetary geographic area having a high release of the gaseous chemical compound.

6. The method of claim 1, wherein the gaseous chemical compound is from a planetary atmosphere, wherein the gaseous chemical compound comprises a noble gas and/or isotopes thereof.

7. The method of claim 1, wherein the planetary gas is from Earth, an astronomical object in the Earth's solar system, or is identified from an analysis of an astronomical object in the Earth solar system, or an extra solar (exo)-planet.

8. The method of claim 1, further comprising the step of preparing a pharmaceutical formulation from said gas marble, including a pharmaceutical formulation that is an oral delivery capsule, a suppository delivery capsule, a topical patch, a foam, a cream, or an intravenously administered formulation.

9. The method of claim 1, further comprising the step of providing a selected dose of said gaseous chemical compound in the gas marble, including the selected dose that is greater than or equal to 1 pg and less than or equal to 10 g.

10. The method of claim 1, wherein the gas marble comprises a biodegradable shell surrounding the gaseous chemical compound, the biodegradable shell comprising a lipid, glyceryl dibehenate, glyceryl distearate, oligosaccharides components, a biopolymer, a functionalized porous silicon nanoparticle, a plant-derived nanoparticle, or a composite material.

11. The method of claim 10, wherein the biodegradable shell comprises a functionalized nanolipid particle with a ligand, wherein the ligand is a peptide, a protein or a nucleic acid.

12. The method of claim 1, wherein the gas marble further comprises a solid excipient for controlled release, including by rapid onset and/or prolonged release.

13. The method of claim 12, wherein the release is by rapid onset induced by pH activation, electromagnetic irradiation, and/or enzymatic digestion.

14. The method of claim 1, wherein the gas marble further comprises an adjuvant.

15. The method of claim 1, wherein the gas marble has a shell configured to withstand a physical force associated with an external environment and/or handling, including the physical force by one or more of:
gravity-induced drainage, liquid evaporation, presence of nuclei, or Laplace overpressure.

16. The method of claim 1, wherein the gaseous chemical compound is associated with a planetary atmosphere undergoing a measurable and quantifiable perturbation, including by climate change, wherein the perturbation is measured and quantified by an aerial, oceanic or satellite measurement.

17. The method of claim 16, further comprising the steps of:
assessing, calculating and mapping outcomes based on said aerial, oceanic or satellite measurement with a mathematical computing algorithm to identify, locate and quantify said gaseous chemical compounds or a precursor thereof in said planetary atmosphere; and
optionally storing said assessed and mapped outcomes onto a computer-readable database.

18. The method of claim 1, further comprising the step of determining an optimal gas release location on a planetary geographic area and timepoint range for collection of the gaseous chemical compound or a precursor thereof from the planetary atmosphere.

19. The method of claim 1, further comprising the steps of:
processing a first chemical compound or mixture thereof from said planetary atmosphere;
applying said first chemical compound or mixture thereof to solubilize a second chemical compound from a planetary crust region and/or an oceanic fluid;
releasing said solubilized second compound into the planetary atmosphere; and
collecting said second chemical compound from the planetary atmosphere using a direct gas collection such as airborne, ship, buoy or other suitable sampling method.

20. The method of claim 1, wherein the gaseous chemical compound comprises NO gas, CO gas, noble gases or isotopes thereof, oxygen, ozone, sulfur-containing compounds, and/or a gaseous species used in a medical gas therapy.

21. The method of claim 1, wherein the biological system is a patient in need of a medical gas therapy, and/or a patient undergoing a multi-modal therapy comprising chemo, radio, cellular, gene, thermo or dynamic therapies, and/or an individual undergoing a physiological performance enhancement.

22. The method of claim 21, wherein the patient suffers from a cancer, an infection, an autoimmune disease, a neurological disease, a cardiovascular disease, a rare disease, a trauma, or a disease-induced environmental exposure, including irradiation.

23. The method of claim 1, wherein the applying step is by oral administration, suppository administration, subcutaneous injection, intramuscular administration, intravenous administration, topical application, or another delivery modality.

24. A stable gas marble made by the method of claim 1, wherein the stable gas marble is configured to maintain size and shape for long term storage ranging from one hour to one year.
